SEQUENCE LISTING (26 sequences)

SEQ ID NO: 1 - DNA, 998 bp, synthetic construct
  misc_feature 1..998: note = Description of sequence: EPOR-integrating cassette left homology arm:
  source 1..998: mol_type = other DNA
gagatgggggtctcactatgtttcctaggctggtcttgaactcctgggttcaaatgatcctcccacctcagcctcccaaagtactgggattatagtgctggtgtaaaccactgcacctggccatggccaggattaaagggagaatgaccaaggtatattgaactcctatgcacccttcaataccctgttccatttacccttttgtagggccttgctgatgcttcagccaaaacccctgtcccctggccctgatgtactcctctgcctccattgtgatcacagggaccaagtgtatctgtgcctctatgactgggagtggagggggaattggtgagtattcaatgagtcatatctatgtaactatttatattggcttcaacagggctctgaagcagaagatctggcctggcatcccgagcccagagagcgagtttgaaggcctcttcaccacccacaagggtaacttccaggtaggtggcctggttgtcccctcagtgcctgggcttccctgcttcttgcagccaaactgcaggcctctctgagcaggttggtgctatttcttcagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatagctcgagggcgcgcccgctgatcagcctcgac

SEQ ID NO: 2 - DNA, 962 bp, synthetic construct
  misc_feature 1..962: note = Description of sequence: EPOR-integrating cassette right homology arm:
  source 1..962: mol_type = other DNA
acactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttttgtatctatatatatatatacacatatgtatgtaagtttttctaccatgatttctacaaacaccctttaagtcccatcttcccctgggcataggccatagggatagaagttaaagttcttgagcttattcagaagctggatctgcaatctgaatgctactcataacataacaaaatagtatgttaaacagctcttaaatcttactggcttaccacattaaatgatttctctctcctaactcagctcaaatgggcagccatccatgggatgagtcagaggttcagactcttccagtctgtagctctaccttctcttagggtacttagatggatcccctgttctacaaactgccagtcagcaagggaagaaaaagggcagcaatgaccctcaatgggccatttgagggatctggcctggaaatgggcttcctctcttcttctcacacctcactggctggaaacagtcacatgaccccagtcacatgaaaggccaggaaacttagtttagctgtacacccaggaagggcaaagctgtttaagggccactagctagtctctgccactaat

SEQ ID NO: 3 - DNA, 2198 bp, synthetic construct
  misc_feature 1..2198: note = Description of sequence: bGH-UbC-GFP cassette that introduces truncation of endogenous locus:
  source 1..2198: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccattaccctgttatccctaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctag

SEQ ID NO: 4 - DNA, 946 bp, synthetic construct
  misc_feature 1..946: note = Description of sequence: HBA1-integrating cassette left homology arm:
  source 1..946: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccacc

SEQ ID NO: 5 - DNA, 879 bp, synthetic construct
  misc_feature 1..879: note = Description of sequence: HBA1-integrating cassette right homology arm:
  source 1..879: mol_type = other DNA
tggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 6 - DNA, 2088 bp, synthetic construct
  misc_feature 1..2088: note = Description of sequence: tEPOR cDNA-T2A-YFP cassette for integration at HBA1:
  source 1..2088: mol_type = other DNA
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccagagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 7 - DNA, 400 bp, synthetic construct
  misc_feature 1..400: note = Description of sequence: CCR5-integrating cassette left homology arm:
  source 1..400: mol_type = other DNA
aagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttc

SEQ ID NO: 8 - DNA, 400 bp, synthetic construct
  misc_feature 1..400: note = Description of sequence: CCR5-integrating cassette right homology arm:
  source 1..400: mol_type = other DNA
tgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaa

SEQ ID NO: 9 - DNA, 3576 bp, synthetic construct
  misc_feature 1..3576: note = Description of sequence: UbC-tEPOR cDNA-T2A-YFP cassette for integration at CCR5:
  source 1..3576: mol_type = other DNA
attaccctgttatccctaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacggtaccgagctcttcgaaggatccatcgccaccatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccagagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - DNA, 1314 bp, synthetic construct
  misc_feature 1..1314: note = Description of sequence: tEPOR cDNA:
  source 1..1314: mol_type = other DNA
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtcca

SEQ ID NO: 11 - DNA, 20 bp, synthetic construct
  misc_feature 1..20: note = Description of sequence: tEpor_sg1 target sequence
  source 1..20: mol_type = other DNA
gctcccagctcttgcgtcca

SEQ ID NO: 12 - DNA, 20 bp, synthetic construct
  misc_feature 1..20: note = Description of sequence: tEpor_sg2 target sequence
  source 1..20: mol_type = other DNA
agctcagggcacagtgtcca

SEQ ID NO: 13 - DNA, 20 bp, synthetic construct
  misc_feature 1..20: note = Description of sequence: HBA1 sg5 target sequence
  source 1..20: mol_type = other DNA
ggcaagaagcatggccaccg

SEQ ID NO: 14 - DNA, 20 bp, synthetic construct
  misc_feature 1..20: note = Description of sequence: CCR5 sg target sequence
  source 1..20: mol_type = other DNA
gcagcatagtgagcccagaa

SEQ ID NO: 15 - DNA, 20 bp, synthetic construct
  misc_feature 1..20: note = Description of sequence: mm_tEpor_sg1
  source 1..20: mol_type = other DNA
ctggaaggtgaggtgccctc

SEQ ID NO: 16 - DNA, 20 bp, synthetic construct
  misc_feature 1..20: note = Description of sequence: mm_tEpor_sg2
  source 1..20: mol_type = other DNA
gtactcaaagctggaaggtg

SEQ ID NO: 17 - DNA, 20 bp, synthetic construct
  misc_feature 1..20: note = Description of sequence: mm_tEpor_sg3
  source 1..20: mol_type = other DNA
gcctcaaagcccaggccaga

SEQ ID NO: 18 - DNA, 20 bp, synthetic construct
  misc_feature 1..20: note = Description of sequence: mm_tEpor_sg4
  source 1..20: mol_type = other DNA
aggtgaggtgccctctggcc

SEQ ID NO: 19 - DNA, 900 bp, synthetic construct
  misc_feature 1..900: note = Description of sequence: HBB left homology arm
  source 1..900: mol_type = other DNA
attagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacacc

SEQ ID NO: 20 - DNA, 900 bp, synthetic construct
  misc_feature 1..900: note = Description of sequence: HBB right homology arm
  source 1..900: mol_type = other DNA
ggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaa

SEQ ID NO: 21 - DNA, 20 bp, synthetic construct
  misc_feature 1..20: note = Description of sequence: HBB sg7 target sequence
  source 1..20: mol_type = other DNA
gggtgggaaaatagaccaat

SEQ ID NO: 22 - DNA, 20 bp, synthetic construct
  misc_feature 1..20: note = Description of sequence: HBB sg11 target sequence
  source 1..20: mol_type = other DNA
tatggttaagttcatgtcat

SEQ ID NO: 23 - DNA, 20 bp, synthetic construct
  misc_feature 1..20: note = Description of sequence: HBB sg13 target sequence
  source 1..20: mol_type = other DNA
taggaaggggataagtaaca

SEQ ID NO: 24 - DNA, 587 bp, synthetic construct
  misc_feature 1..587: note = Description of sequence: IRES sequence
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 25 - DNA, 78 bp, synthetic construct
  misc_feature 1..78: note = Description of sequence: T2A cleavage peptide
  source 1..78: mol_type = other DNA
cgcaagcgccgcagcggcagcggcgagggccgcggcagcctgctgacctgcggcgacgtggaggagaaccccggcccc

SEQ ID NO: 26 - DNA, 78 bp, synthetic construct
  misc_feature 1..78: note = Description of sequence: P2A cleavage peptide
  source 1..78: mol_type = other DNA
cgcaaaagacgctccggttctggagagggcagggggagtcttcttacatgcggggatgttgaggagaatcccggaccc

(19) United States
(12) Patent Application Publication
(10) Pub. No.: US 2024/0409958 A1
(43) Pub. Date: Dec. 12, 2024

(54) DIFFERENTIAL PROLIFERATION OF HUMAN HEMATOPOIETIC STEM AND PROGENITOR CELLS USING TRUNCATED ERYTHROPOIETIN RECEPTORS

(71) Applicant: The Board of Trustees of the Leland Stanford Junior University, Stanford, CA (US)

(72) Inventors: Matthew H. Porteus, Stanford, CA (US); Michael Kyle Cromer, Stanford, CA (US); Joab Camarena, Stanford, CA (US)

(73) Assignee: The Board of Trustees of the Leland Stanford Junior University, Stanford, CA (US)

(21) Appl. No.: 18/700,219

(22) PCT Filed: Oct. 12, 2022
(86) PCT No.: PCT/US2022/077951

§ 371 Date: Apr. 10, 2024

Related U.S. Application Data
(60) Provisional application No. 63/308,914, filed on Feb. 10, 2022.
(60) Provisional application No. 63/255,412, filed on Oct. 13, 2021.

(51) Int. Cl. C12N 15/864; C12N 5/0789; C12N 9/22; C12N 15/11

(52) U.S. Cl. CPC C12N 15/8645; C12N 5/0647; C12N 9/22; C12N 15/11; C12N 2310/20; C12N 2510/00; C12N 2750/14143; C12N 2800/60

(57) ABSTRACT

Edited cell chimerism is currently one of the greatest bottlenecks to clinical efficacy of gene therapies for the hemoglobinopathies. For example, it is difficult to go from low hematopoietic stem cell (HSC) edited cell chimerism in the bone marrow to high edited red blood cell (RBC) chimerism in the bloodstream. The present disclosure provides methods and compositions for genetically modifying hematopoietic stem and progenitor cells (HSPCs), in particular by creating HSPCs that express truncated forms of the EPO.receptor (tEPOR).

### CROSS-REFERENCE TO RELATED APPLICATIONS

This application claims priority to U.S. Provisional Application No. 63/255,412, filed Oct. 13, 2021, and U.S. Provisional Application No. 63/308,914, filed Feb. 10, 2022, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. HL135607 awarded by the National Institutes of Health. The Government has certain rights in the invention.

### BACKGROUND

Edited cell chimerism is currently one of the greatest bottlenecks to clinical efficacy of gene therapies for the hemoglobinopathies. For example, it is difficult to go from low hematopoietic stem cell (HSC) edited cell chimerism in the bone marrow to high edited red blood cell (RBC) chimerism in the bloodstream. As a result, transplanted genetically modified HSCs comprising, e.g., corrected mutant genes or exogenous transgenes encoding therapeutic proteins, often fail to proliferate or be maintained sufficiently to make up a high enough proportion of erythroid cells in an individual to provide an effective therapeutic benefit.

Primary familial polycythemia, also referred to as benign erythrocytosis, is an inherited hematological disorder in which the body produces an elevated number of RBCs. The increased number of RBCs is a result of an increase in the activity of signaling pathways downstream of the erythropoietin (EPO) receptor, even in the presence of low levels of EPO. This increased signaling activity results in the enhanced proliferation, survival, and/or differentiation of cells in the erythroid lineage, resulting in the overproduction of RBCs. Primary familial polycythemia can involve erythrocytosis but without any other phenotypic abnormalities. For example, the Finnish cross-country skier and Olympic medalist Eero Mäntyranta was found to have remarkably high levels of hemoglobin even with low levels of EPO. In a study of an extended family in Finland, researchers found erythrocytosis to be inherited as a dominant trait, caused by a nonsense mutation in the gene encoding the EPO receptor (EPOR) that resulted in a 70 amino acid truncation of the receptor.

EPOR is a member of the cytokine receptor family cytokine that regulates the proliferation and differentiation of erythroid precursor cells. Upon binding of EPO, EPOR activates JAK2 tyrosine kinase, which in turn activates different intracellular pathways such as Ras/MAP kinase. PI3 kinase, and STAT transcription factors. Normally, the activation of EPOR signaling pathways is inhibited by the intracellular binding of the protein tyrosine phosphatase SHP-1. Truncated forms of the EPOR that underlie primary familial polycythemia, however, can lack the SHP-1 binding site, resulting in EPO hypersensitivity and enhanced EPOR signaling activity.

There is therefore a need for new methods and compositions that can safely and effectively enhance the chimerism of edited RBCs in subjects. The present disclosure satisfies this need and provides other advantages as well.

### BRIEF SUMMARY

In one aspect, the present disclosure provides a method of genetically modifying a hematopoietic stem and progenitor cell (HSPC), the method comprising: introducing into the HSPC an RNA-guided nuclease and a guide RNA that specifically targets a sequence within the cytoplasmic domain-encoding region of the erythropoietin receptor (EPOR) locus in the genome of the cell; wherein the RNA-guided nuclease cleaves the EPOR locus in the genome of the cell, resulting in the expression of a truncated erythropoietin receptor (tEPOR) in the cell and thereby generating a genetically modified HSPC; and wherein the expression of the tEPOR increases the sensitivity of the cell to erythropoietin (EPO) and/or increases the proliferation of the cell in the presence of EPO as compared to the sensitivity and/or proliferation of a non-genetically modified HSPC.

In some embodiments, the cleavage of the EPOR locus by the RNA-guided nuclease creates an insertion or deletion (indel) that introduces a nonsense mutation into the EPOR locus. In some embodiments, the method further comprises introducing a donor template into the cell, wherein the donor template comprises a first homologous region comprising complementarity to the EPOR locus upstream of the guide RNA target site, a second homologous region comprising complementarity to the EPOR locus downstream of the guide RNA target site, and a coding sequence located between the first and second homology regions that encodes a tEPOR, and wherein the coding sequence is integrated into the cleaved EPOR locus, leading to the expression of the tEPOR in the cell.

In some embodiments, the method further comprises isolating the HSPC from a subject prior to introducing the RNA-guided nuclease and the guide RNA into the cell. In some embodiments, expression of the tEPOR is driven by the endogenous EPOR promoter. In some embodiments, the tEPOR lacks a C-terminal portion of the EPOR cytoplasmic domain. In some embodiments, the first homology arm comprises the nucleotide sequence of SEQ ID NO:1 or a subsequence thereof, or a sequence comprising at least about 70%, 75%, 80%, 85%, 90%, 95%, 96%, 97%, 98%, 99%, or more identity to SEQ ID NO:1 or a subsequence thereof. In some embodiments, the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof.

In some embodiments, the coding sequence encoding the truncated EPOR protein comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10. In some embodiments, the donor template comprises SEQ ID NO:3 or a subsequence thereof, or a sequence comprising at least about 70%, 75%, 80%, 85%, 90%, 95%, 96%, 97%, 98%, 99% or more identity to SEQ ID NO:3 or a subsequence thereof. In some embodiments, the target sequence of the guide RNA comprises the nucleotide sequence of SEQ ID NO: 11 or 12, or a nucleotide sequence comprising 1, 2, or 3 mismatches relative to SEQ ID NO: 11 or 12.

In some embodiments, the guide RNA comprises one or more 2′-O-methyl-3′-phosphorothioate (MS) modifications. In some embodiments, the one or more 2′-O-methyl-3′-phosphorothioate (MS) modifications are present at the three terminal nucleotides of the 5′ and 3′ ends of the guide RNA. In some embodiments, the RNA-guided nuclease is Cas9. In some embodiments, the Cas9 is a High Fidelity Cas9. In some embodiments, the guide RNA and the RNA-guided nuclease are introduced into the HSPC as a ribonucleoprotein (RNP) complex by electroporation. In some embodiments, the donor template is introduced into the HSPC using a recombinant adeno-associated virus (rAAV) vector. In some embodiments, the rAAV vector is a AAV6 vector.

In some embodiments, the HSPC is genetically modified at a second locus other than EPOR, using an sgRNA targeting the second locus and a second homologous donor template comprising homology to the second locus. In some embodiments, the second homologous donor template further comprises a therapeutic transgene. In some embodiments, the therapeutic transgene is selected from the group consisting of HBA1, HBA2, HBB, PDGFB. IDUA, FIX, LDLR, and PAH. In some embodiments, the HSPC is isolated from a subject having a condition for which the genetic modification made at the second locus is beneficial. In some embodiments, the condition is α-thalassemia, β-thalassemia, sickle cell disease, hemophilia B, phenylketonuria, Gaucher disease, or Krabbe disease. In some embodiments, the genetically modified HSPC is reintroduced into the subject. In some embodiments, the reintroduction of the genetically modified HSPC ameliorates one or more symptoms of the condition. In some embodiments, the proportion of genetically modified HSPCs among red blood cells (RBCs) and/or one or more myeloid or lymphoid lineages in the subject increases over time. In some embodiments, the subject is a human.

In another aspect, the present disclosure provides a method of genetically modifying a hematopoietic stem and progenitor cell (HSPC), the method comprising: introducing into the HSPC an RNA-guided nuclease, a donor template comprising a transgene encoding a truncated erythropoietin receptor (tEPOR), and a guide RNA that specifically targets a sequence within a safe harbor locus in the genome of the cell, wherein the donor template comprises a first homologous region comprising complementarity to the safe harbor locus upstream of the guide RNA target site, a second homologous region comprising complementarity to the safe harbor locus downstream of the guide RNA target site, wherein the first and second homology regions flank the tEPOR transgene on the template, wherein the RNA-guided nuclease cleaves the safe harbor locus in the genome of the cell and the transgene is integrated into the genome at the cleaved safe harbor locus, thereby generating a genetically modified HSPC; and wherein the integrated transgene results in expression of the tEPOR in the genetically modified HSPC.

In some embodiments, expression of the tEPOR increases the sensitivity of the cell to erythropoietin (EPO) and/or increases the proliferation of the cell in the presence of EPO relative to the sensitivity and/or proliferation of a non-genetically modified HSPC. In some embodiments, the method further comprises isolating the HSPC from a subject prior to introducing the RNA-guided nuclease, the donor template, and the guide RNA into the cell. In some embodiments, the transgene encoding the truncated EPOR comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10. In some embodiments, the guide RNA comprises one or more 2′-O-methyl-3′-phosphorothioate (MS) modifications. In some embodiments, the one or more 2′-O-methyl-3′-phosphorothioate (MS) modifications are present at the three terminal nucleotides of the 5′ and 3′ ends of the guide RNA. In some embodiments, the RNA-guided nuclease is Cas9. In some embodiments, the Cas9 is a High Fidelity Cas9. In some embodiments, the guide RNA and the RNA-guided nuclease are introduced into the HSPC as a ribonucleoprotein (RNP) complex by electroporation. In some embodiments, the donor template is introduced into the HSPC using a recombinant adeno-associated virus (rAAV) vector. In some embodiments, the rAAV vector is a AAV6 vector.

In some embodiments, the safe harbor locus is CCR5. In some such embodiments, the first homology arm comprises the nucleotide sequence of SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof. In some embodiments, the second homology arm comprises the nucleotide sequence of SEQ ID NO:8 or a subsequence thereof, or a sequence comprising at least about 70%, 75%, 80%, 85%, 90%, 95%, 96%, 97%, 98%, 99%, or more identity to SEQ ID NO:8 or a subsequence thereof. In some embodiments, the target sequence of the guide RNA comprises the nucleotide sequence of SEQ ID NO: 14, or a nucleotide sequence comprising 1, 2, or 3 mismatches relative to SEQ ID NO: 14. In some embodiments, the donor template comprises SEQ ID NO:9 or a subsequence thereof, or a sequence comprising at least about 70%, 75%, 80%, 85%, 90%, 95%, 96%, 97%, 98%, 99% or more identity to SEQ ID NO:9 or a subsequence thereof.

In some embodiments, the safe harbor locus is HBA1. In some such embodiments, the first homology arm comprises the nucleotide sequence of SEQ ID NO:4 or a subsequence thereof, or a sequence comprising at least about 70%, 75%, 80%, 85%, 90%, 95%, 96%, 97%, 98%, 99%, or more identity to SEQ ID NO:4 or a subsequence thereof. In some embodiments, the second homology arm comprises the nucleotide sequence of SEQ ID NO:5 or a subsequence thereof, or a sequence comprising at least about 70%, 75%, 80%, 85%, 90%, 95%, 96%, 97%, 98%, 99%, or more identity to SEQ ID NO:5 or a subsequence thereof. In some embodiments, the target sequence of the guide RNA comprises the nucleotide sequence of SEQ ID NO: 13, or a nucleotide sequence comprising 1, 2, or 3 mismatches relative to SEQ ID NO: 13. In some embodiments, the donor template comprises SEQ ID NO:6 or a subsequence thereof, or a sequence comprising at least about 70%, 75%, 80%, 85%, 90%, 95%, 96%, 97%, 98%, 99% or more identity to SEQ ID NO:6 or a subsequence thereof. In some embodiments, the donor template further comprises a therapeutic transgene encoding a protein, and wherein the first and second homology regions flank the therapeutic transgene and the tEPOR transgene on the template. In some embodiments, the donor template further comprises an internal ribosome entry site (IRES) or a sequence encoding a 2A cleavage peptide between the therapeutic transgene and the tEPOR transgene on the template. In some embodiments, the HSPC comprises a mutation in an endogenous gene causative of a condition in a subject and the therapeutic transgene comprises a corrective sequence. In some embodiments, the therapeutic transgene is selected from the group consisting of HBB, PDGFB, IDUA, FIX, LDLR, and PAH. In particular embodiments, the therapeutic transgene is HBB and the condition is β-thalassemia or sickle cell disease. In particular embodiments, the therapeutic transgene is FIX and the condition is hemophilia B. In particular embodiments, the therapeutic transgene is PAH and the condition is phenylketonuria.

In some embodiments, the safe harbor locus is HBB. In some such embodiments, the first homology arm comprises the nucleotide sequence of SEQ ID NO:19 or a subsequence thereof, or a sequence comprising at least about 70%, 75%, 80%, 85%, 90%, 95%, 96%, 97%, 98%, 99%, or more identity to SEQ ID NO:19 or a subsequence thereof. In some embodiments, the second homology arm comprises the nucleotide sequence of SEQ ID NO:20 or a subsequence thereof, or a sequence comprising at least about 70%, 75%, 80%, 85%, 90%, 95%, 96%, 97%, 98%, 99%, or more identity to SEQ ID NO:20 or a subsequence thereof. In some embodiments, the target sequence of the guide RNA comprises the nucleotide sequence of SEQ ID NO: 21, or a nucleotide sequence comprising 1, 2, or 3 mismatches relative to SEQ ID NO: 21. In other embodiments, the target sequence of the guide RNA comprises the nucleotide sequence of SEQ ID NO: 22 or 23, or a nucleotide sequence comprising 1, 2, or 3 mismatches relative to SEQ ID NO: 22 or 23. In some embodiments, the donor template further comprises a therapeutic transgene encoding a protein, and wherein the first and second homology regions flank the therapeutic transgene and the tEPOR transgene on the template. In some embodiments, the donor template further comprises an internal ribosome entry site (IRES) or a sequence encoding a 2A cleavage peptide between the therapeutic transgene and the tEPOR transgene on the template. In some embodiments, the HSPC comprises a mutation in an endogenous gene causative of a condition in a subject and the therapeutic transgene comprises a corrective sequence. In some embodiments, the therapeutic transgene is HBA1 or HBA2. In some embodiments, the condition is α-thalassemia. In particular embodiments, the therapeutic transgene is HBA1 and the condition is α-thalassemia.

In some embodiments, the HSPC comprises a population of HSPCs. In some embodiments, expression of the therapeutic transgene and the tEPOR transgene causes an enrichment of genetically modified HSPCs in the population of HSPCs over the course of red blood cell differentiation as compared to expression of the therapeutic transgene in the absence of expression of the tEPOR transgene. In some embodiments, expression of the therapeutic transgene and the tEPOR transgene increases a level of adult hemoglobin tetramers in the genetically modified HSPC as compared to expression of the therapeutic transgene in the absence of expression of the tEPOR transgene.

In some embodiments, the HSPC is genetically modified at a second locus other than the safe harbor locus using an sgRNA targeting the second locus and a second homologous donor template comprising homology to the second locus. In some embodiments, the second homologous donor template further comprises a therapeutic transgene. In some embodiments, the therapeutic transgene is selected from the group consisting of HBA1, HBA2, HBB, PDGFB, IDUA, FIX, LDLR, and PAH.

In some embodiments, the HSPC is isolated from a subject having a condition for which the genetic modification introduced at the second locus is beneficial. In some such embodiments, the genetically modified HSPC is reintroduced into the subject. In some embodiments, the reintroduction of the genetically modified HSPC ameliorates one or more symptoms of the condition. In some embodiments, the proportion of genetically modified HSPCs among red blood cells (RBCs) and/or one or more myeloid or lymphoid lineages in the subject increases over time following the reintroduction of the HSPC into the subject. In some embodiments, the therapeutic transgene and/or tEPOR transgene comprises a heterologous promoter. In some embodiments, the heterologous promoter is selected from the group consisting of EPOR, HBA1, PGK1, and UBC. In other embodiments, the therapeutic transgene and/or tEPOR transgene is under the control of the endogenous promoter of the safe harbor locus, e.g., the HBA1 or HBB promoter. In some embodiments, the condition is α-thalassemia, β-thalassemia, sickle cell disease, hemophilia B, phenylketonuria, Gaucher disease, or Krabbe disease. In some embodiments, the subject is a human.

In another aspect, the present disclosure provides a genetically modified HSPC comprising a coding sequence encoding a tEPOR, wherein the genetically modified HSPC is generated using any of the herein-described methods.

In another aspect, the present disclosure provides a donor template comprising a homology region comprising SEQ ID NO: 1 or SEQ ID NO: 2 or a subsequence thereof, or a nucleotide sequence comprising at least about 70%, 75%, 80%, 85%, 90%, 95%, 96%, 97%, 98%, 99% or more identity to SEQ ID NO: 1 or SEQ ID NO: 2 or a subsequence thereof.

In another aspect, the present disclosure provides a donor template comprising a nucleotide sequence selected from the group consisting of SEQ ID NOS: 3, 6, and 9 and subsequences thereof, or a nucleotide sequence comprising at least about 70%, 75%, 80%, 85%, 90%, 95%, 96%, 97%, 98%, 99% or more identity to any one of SEQ ID NOS: 3, 6, or 9, or a subsequence thereof.

In another aspect, the present disclosure provides a transgene comprising a nucleotide sequence encoding a tEPOR, wherein the nucleotide sequence comprises the sequence of SEQ ID NO:10, or a nucleotide sequence comprising at least about 70%, 75%, 80%, 85%, 90%, 95%, 96%, 97%, 98%, 99% or more identity to SEQ ID NO:10.

In another aspect, the present disclosure provides a guide RNA comprising a target sequence comprising SEQ ID NO: 11 or SEQ ID NO:12, or a sequence comprising 1, 2, or 3 mismatches with SEQ ID NO: 11 or SEQ ID NO:12.

In another aspect, the present disclosure provides an HSPC comprising any of the herein described donor templates, transgenes, and/or guide RNAs.

### BRIEF DESCRIPTION OF THE DRAWINGS

FIGS. 1A-IC: Two SpCas9 gRNAs (FIG. 1A) were designed overlapping the variant responsible for the truncation in the Olympic cross-country skier Eero Mäntyranta. This was expected to yield an indel spectrum, a subset of which were expected to yield premature truncation of the endogenous EPOR. These gRNAs were pre-complexed with high fidelity variant SpCas9 RNP (Vakulskas, et al. Nature Medicine, 2018) and delivered by electroporation to WT HSPCs. We then performed a three-phase RBC differentiation protocol and harvested genomic DNA over the course of the 16d protocol to determine whether enrichment of HSPCs with indels occurred (FIG. 1B). Because the vast majority of editing has occurred by 24-48 h, any enrichment or depletion of edited cells following this is likely to represent a functional consequence of editing. We observed ˜4× enrichment of the edited cells occurring rapidly between d1 and d9, which was maintained throughout the remainder of RBC differentiation (FIG. 1C).

FIGS. 2A-2C: We tested whether the enrichment effect is limited to cells that are erythroid-differentiated, or whether enrichment also occurs in edited HSPCs maintained in HSC media (FIG. 2A). We found that enrichment occurred in both conditions (FIGS. 2B-2C), though the effect was more immediate in cells that underwent RBC differentiation (FIG. 2C).

FIGS. 3A-3C: The amounts of Cas9 and gRNA used were titrated in order to determine the degree of enrichment that occurs following lower initial editing frequencies. We found enrichment (from ˜30% to ˜70% indels) in standard conditions (450 mg/mL Cas9) over the course of RBC differentiation, which was slightly less pronounced in ⅓ condition and virtually absent in 1/9 Cas9 condition. FIG. 3B shows the frequency of indels over the course of erythroid differentiation from a single treatment targeted with tEPOR sg2, but maintained in either standard HSC media or RBC differentiation media with 0, 3, or 20 U/mL EPO. Note, in normal RBC differentiation, 3 U/mL of EPO is maintained through the 14d protocol. Here, we found that enrichment occurred in all groups, though it was most evident in the 3 and 20 U/mL EPO conditions. FIG. 3C: editing with these EPOR-truncating gRNAs had no negative impact on the ability of cells to differentiate down the erythroid lineage.

FIGS. 4A-4E: To determine which indels were driving the observed enrichment (for this sample, from 27.5% indels at d1 to 67.7% indels at d14; FIG. 4A), we plotted the five most common indels found in this treatment (WT HSPCs targeted with tEPOR sg2) over the course of RBC differentiation (FIG. 4B). The distribution of the indels at day 1 (FIG. 4C) and at day 14 (FIG. 4D) are also shown. We found that of the five most common indels, the four that introduce downstream truncation events undergo the most enrichment compared to the single indel (a 6 bp deletion) which would maintain the reading frame, and therefore not lead to a truncation (FIG. 4E).

FIGS. 5A-5E: The same data as plotted in FIGS. 4A-4E, but for WT HSPCs targeted with tEPOR sg1. Again, of the five most common indels, the four that lead to truncation are enriched (FIG. 5E), while the single indel that maintains the reading frame (a 6 bp deletion) is fully depleted by d14 (FIG. 5D).

FIGS. 6A-6C: To further investigate the cell types capable of undergoing enrichment, we targeted WT HSPCs as before with tEPOR sg2, performed erythroid differentiation, and then at d16 sorted out cells that both had and had not acquired erythroid markers (CD71 and GPA) (FIG. 6A). We found that while the frequency of indels is enriched in both cell populations, this effect is most pronounced in the cells that have more fully differentiated down the RBC lineage (FIG. 6B). To investigate whether this effect may be driven by EPOR expression, we determined mRNA expression harvested from d16 RBCs by ddPCR and found that EPOR expression directly correlated with GPA expression (FIG. 6C).

FIG. 7: We designed a polyA-UbC-GFP integration cassette that could be delivered by AAV6 transduction following cleavage by tEPOR sg2, in order to yield a population of GFP+ cells that all would have at least a heterozygous truncation of EPOR. This would allow us to measure GFP+ in real-time as cells differentiate into RBCs. In addition to methods 1 and 2, which alter the endogenous EPOR (which is relatively poorly expressed), methods 3 and 4 introduce a tEPOR cDNA as a transgene. The transgene could be driven by an endogenous promoter, like the HBA1 erythroid-specific safe harbor site (method 4). We could also deliver this tEPOR transgene into a typical safe harbor site like CCR5 which would be driven by a custom strong constitutive promoter like UbC (method 3).

FIG. 8: Because constructs 2-4 in FIG. 7 all have fluorescent markers that integrate along with tEPOR, we can determine enrichment in the various cell populations in real-time over the course of erythroid differentiation using flow cytometry.

FIG. 9: 2 days post-editing, HSPCs targeted with the constructs shown were plated in RBC differentiation media containing either 0 or 3 U/mL EPO (top row and bottom row, respectively). The percentage of GFP+ cells was plotted in the more stem-like population (CD34+/CD45+/CD71−/GPA−) as well as the population that acquired RBC-specific markers (CD34−/CD45−/CD71+/GPA+). As expected, the most dramatic enrichment occurred only in the presence of EPO in the RBC population. In spite of strong and/or constitutive expression of tEPOR by the HBA1- and CCR5-integrating constructs, very little enrichment occurred in the absence of EPO or in the stem-like population in the presence of EPO. The percentage of YFP+ cells is indicated as “% GFP*” for the constructs in the middle and on the right because GFP and YFP are in the same “FITC” channel on flow cytometry.

FIGS. 10A-10C: To confirm that editing frequencies did in fact increase over the course of RBC differentiation, ddPCR was used to quantify editing frequencies in the 0 and 3 U/mL EPO conditions depicted in FIG. 9. Dramatic enrichment of edited cells was observed with all three constructs.

FIG. 10D: Cells were edited as shown in FIG. 8 with the HBA1-integrating tEPOR cassette. At d14 of RBC differentiation, we analyzed cells by FACS and observed that YFP (indicated as “GFP+”) was only expressed in cells that had acquired erythroid-specific markers (CD71+/GPA+).

FIGS. 10E-10F: To determine the impact of tEPOR introduction on hemoglobin production, we edited cells as depicted in FIG. 1B and at d14 of RBC differentiation, harvested cell pellets for hemoglobin tetramer HPLC analysis. We found that cells edited with both the endogenous EPOR-truncating gRNA “sg2” (FIG. 10E) as well as the HBA1-integrating tEPOR cassette (FIG. 10F) are able to yield both fetal and adult hemoglobin (HbF and HbA, respectively).

FIG. 11: To determine the impact of tEPOR introduction on lineage bias and colony-forming ability of edited HSPCs, we performed CFU assays. In the presence or absence of EPO in the methylcellulose, we observed no substantial lineage bias in any of the edited conditions. While cells edited for a non-tEPOR transgene (FIX gene for correction of hemophilia B, “Padua”) showed a dramatic decrease in colony-forming ability, this effect was not observed with any of the tEPOR-integrating constructs.

FIGS. 12A-12B: Four gRNAs were screened that introduced indels nearby the reported functional truncation of the endogenous mouse Epor gene (FIG. 12A). SpCas9 pre-complexed with each gRNA was delivered to mouse HSPCs, and 4d later gDNA was harvested and amplicons surrounding the predicted cleavage sites were Sanger sequenced. Sanger traces were then used as input to TIDE: we found sg1 initiated the highest frequency of indels at this locus (FIG. 12B).

FIG. 13 depicts a large-scale competitive HSC transplantation experiment of edited donor mouse HSCs into recipient mice. The workflow is comprised of HSC harvest from the bone marrow of GFP+ or GFP− B16 mice. A two-week HSC expansion is used to generate a large number of HSCs for editing and subsequent transplantation. Expanded HSCs are then edited with either the functional tEpor gRNA or the non-functional editing at the well-characterized Rosa26 locus. Competitive transplants are next performed by mixing GFP+ and GFP− cells that either do or do not have Epor truncations, and the following metrics are determined over the course of 16 weeks post-transplantation: percentage of GFP+ cells in RBCs and other myeloid and lymphoid lineages, hemoglobin concentration, EPO concentration, blood counts, and clotting ability. At the conclusion of the study, bone marrow is harvested from the mice and the percent engraftment of GFP+ and GFP− populations in phenotypic HSCs and the various lineages is determined.

FIGS. 14A-14D: Schematic for a β-thalassemia (“b-thal”) correction vector that replaces the entire HBA1 gene with a full-length HBB transgene and a bicistronic cassette combining the b-thal correction vector with a tEPOR cDNA is shown (FIG. 14A). Cas9 gRNA #5 is denoted by a star and dotted lines indicate the regions corresponding to the homology arms of the AAV6 donor vector. An internal ribosome entry site (IRES) links the expression of the full-length HBB transgene and the tEPOR cDNA, which are both under the control of the HBA1 promoter for RBC-specific expression. FIG. 14B: Wild-type (WT) primary human CD34+ HSPCs were edited using the schematic depicted in FIG. 14A and differentiated in vitro into RBCs. Genomic DNA was analyzed for editing frequency at days 0, 4, 7, 11, and 14 of the RBC differentiation by ddPCR. The addition of the tEPOR cDNA caused a dramatic enrichment over the course of differentiation. N=3 WT HSPC donors. FIG. 14C: Using sickle cell disease (SCD) HSPC donors and the protocol described in FIG. 14B, we observed a dramatic enrichment in the cells edited with the bicistronic HBB+ tEPOR cassette. N=3 SCD HSPC donors. FIG. 14D: RBC pellets from the edited cells of FIG. 14C were analyzed for hemoglobin tetramer formation by HPLC. The ratio of adult hemoglobin tetramers (HbA) to sickle hemoglobin tetramers (HbS) was plotted. The correction vector alone substantially improved formation of HbA, which was further boosted by the addition of the tEPOR cDNA to the bicistronic vector. N=2 SCD HSPC donors. The “b-thal+ tEPOR” data was generated from replicates from the 2 SCD HSPC donors that were edited separately with the bicistronic HBB+ tEPOR cassette.

FIGS. 15A-15B: Schematic for an α-thalassemia (“a-thal”) correction vector containing a full-length HBA1 transgene that integrates at the start codon of HBB gene and a bicistronic cassette combining the a-thal correction vector with a tEPOR cDNA is shown (FIG. 15A). Cas9 gRNA #7 is denoted by a star and dotted lines indicate the regions corresponding to the homology arms of the AAV6 donor vector. An internal ribosome entry site (IRES) links the expression of full-length HBA1 transgene and the tEPOR cDNA, which are both under the control of the HBB promoter for RBC-specific expression. FIG. 15B: Wild-type (WT) primary human CD34+ HSPCs were edited using the schematic depicted in FIG. 15A and differentiated in vitro into RBCs. Genomic DNA was analyzed for editing frequency at days 0, 4, 7, 11, and 14 of the RBC differentiation by ddPCR. The addition of the tEPOR cDNA caused a dramatic enrichment over the course of differentiation. N=4 WT HSPC donors.

### DETAILED DESCRIPTION

### 1. Introduction

The present disclosure provides methods and compositions for genetically modifying hematopoietic stem and progenitor cells (HSPCs) so as to effect the expression of truncated forms of the erythropoietin receptor (tEPOR). The expression of tEPOR in the modified cells and their descendants, in particular in the erythropoietic lineage (e.g., red blood cells), leads to their enhanced growth and/or proliferation relative to otherwise equivalent HSPCs or HSPC descendants that do not express a tEPOR. This enhanced growth and/or proliferation leads to the enrichment of the cells relative to unmodified cells in vitro and/or in vivo. Accordingly, when coupled with a desired trait or an additional genetic modification (e.g., to effect the expression of a therapeutic transgene), the induction of tEPOR expression in the modified cells can be used to enhance the presence of the desired and/or modified cells in vitro or in vivo.

The present disclosure provides multiple methods for introducing truncated forms of EPOR into cells. For example, in some embodiments an endogenous EPOR gene is modified so as to introduce a nonsense mutation that leads to the truncation of the encoded receptor. In some such embodiments, a guide RNA that targets the EPOR gene is introduced into cells together with an RNA-guided nuclease such as Cas9 so as to cleave the EPOR gene and introduce indels (insertions or deletions) at the cleavage site that often create nonsense mutations. In some embodiments, a guide RNA and RNA-guided nuclease are introduced together with a homologous donor template encoding a truncated form of EPOR, such that the EPOR gene is cleaved and subsequently repaired using the donor template. In some embodiments, transgenes encoding truncated forms of EPOR, e.g., coding sequences with optional elements such as promoters or other regulatory elements (e.g., enhancers, repressor domains), introns, WPREs, poly A regions, UTRs (e.g., 3′ UTRs), are introduced into the genome of the HSPC, e.g., at an endogenous EPOR locus or elsewhere, e.g., a safe-harbor locus such as CCR5, HBA1, or HBB.

The present disclosure provides guide RNA sequences that specifically recognize target sites within the EPOR gene, e.g., within a coding sequence encoding the cytoplasmic domain of the receptor. The present disclosure also provides homologous repair templates encoding truncated forms of EPOR, surrounded by homologous regions comprising homology to the EPOR gene in the proximity of a guide RNA target site. By cleaving EPOR in the presence of a donor template, the tEPOR coding sequence present on the template can be integrated into the genome at the site of cleavage by homology directed recombination (HDR). Expression constructs, vectors, and genetically modified host cells are also provided, as are kits comprising, inter alia, any of the herein-described guide RNAs, donor templates, RNPs, CRISPR/Cas systems, vectors, constructs, pharmaceutical compositions, and/or modified cells.

In particular embodiments, the present methods can be used to enhance the relative presence of another genetic modification among HSPCs and their descendants in a subject. For example, the methods and compositions can be used to enhance the presence of HSPCs comprising beneficial modifications in a subject (e.g., modifications comprising the correction of deleterious mutations in erythroid cells) and/or comprising transgenes whose expression in erythroid cells is beneficial to a subject (e.g., by providing a protein that is deficient in the subject in the absence of the modified cells) using a donor template comprising a therapeutic transgene and a tEPOR transgene as described herein.

### 2. General

Practicing this disclosure utilizes routine techniques in the field of molecular biology. Basic texts disclosing the general methods of use in this disclosure include Sambrook and Russell, Molecular Cloning. A Laboratory Manual(3rd ed. 2001): Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett, 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an.” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11 X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,”-peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a tEPOR encoding mRNA or an encoded tEPOR protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments: or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sc. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types. I, II, III, IV, V, and VI as well as many sub-types, with Class 1 including types I and III CRISPR systems, and Class 2 including types II, IV, V and VI: Class I subtypes include subtypes I-A to I-F, for example. See, e.g., Fonfara et al., Nature 532, 7600 (2016); Zetsche et al., Cell 163, 759-771 (2015): Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” or “donor template” refers to a polynucleotide sequence that can be used to repair a double stranded break (DSB) in the DNA, e.g., a CRISPR/Cas9-mediated break at an EPOR locus as induced using the herein-described methods and compositions. The homologous repair template comprises homology to the genomic sequence surrounding the DSB, i.e., comprising EPOR homology arms. In particular embodiments, two distinct homologous regions are present on the template, with each region comprising at least 50, 100, 200, 300, 400, 500, 600, 700, 800, 900, 400-1000, 500-900, or more nucleotides of homology with the corresponding genomic sequence. In particular embodiments, the templates comprise two homology arms comprising, e.g., about 900 nucleotides of homology, with one arm extending upstream starting at the translation start site, and the other arm extending downstream from the sgRNA target site. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure can also comprise a transgene, e.g., a tEPOR transgene and optionally a therapeutic transgene as described herein.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments, HR involves double-stranded breaks induced by CRISPR-Cas9.

EPOR (erythropoietin receptor) is the receptor for erythropoietin (EPO), a cytokine that regulates the proliferation and differentiation of erythroid precursor cells. When italicized (i.e., EPOR), EPOR refers to a polynucleotide (e.g., gene, locus, transgene, coding sequence, cDNA, expression cassette) encoding EPOR. Upon binding of EPO. EPOR activates JAK2 tyrosine kinase, which in turn activates different intracellular pathways such as Ras/MAP kinase, PI3 kinase, and STAT transcription factors. EPOR is a member of the cytokine receptor family, and the EPOR gene is located on human chromosome 19p (19p13.2). The NCBI gene ID for human EPOR is 2057, and the UniProt ID for human EPOR is P19235, the entire disclosures of which are herein incorporated by reference.

Truncated EPOR, or tEPOR (encoded by tEPOR), refers to forms of the EPO receptor, or to polynucleotides encoding the receptor forms, that lack a portion or all of the receptor's cytoplasmic domain. For example, in some embodiments a tEPOR lacks the 70 C-terminal amino acids of full-length EPOR. In some embodiments, a tEPOR lacks all 236 amino acids of the cytoplasmic domain. In some embodiments, a tEPOR lacks, e.g., about 10, 20, 30, 40, 50, 60, 70, 80, 90, 100, 110, 120, 130, 140, 150, 160, 170, 180, 190, 200, 210, 220, 230, 10-236, 10-50, 50-60, 60-70, 65-75, 70-80, 80-90, 90-100, 100-150, 150-200, or 200-236 amino acids. In some embodiments, a tEPOR lacks a binding site and/or does not interact with the tyrosine phosphatase SHP-1 (or SHPTP-1), which normally plays a role in inhibiting EPOR signaling. In some embodiments, a coding sequence (e.g., gene or transgene) encoding a tEPOR comprises a nonsense mutation in exon 7 or exon 8, and/or encodes any of the herein-described forms of truncated EPOR. Nonsense mutations causing the expression of truncated EPOR act as dominant mutations that render cells hypersensitive to EPO, leading to an ability to undergo effective proliferation and differentiation in the presence of reduced amounts of EPO, and to show enhanced levels of proliferation and differentiation in the presence of normal EPO levels. An exemplary tEPOR cDNA is shown herein as SEQ ID NO:10. As used herein, tEPOR can refer to any nucleotide sequence comprising about 70%, 75%, 80%, 85%, 90%, 95%, 96%, 97%, 98%, 99%, or more homology to SEQ ID NO:10 or a subsequence thereof.

As used herein, the terms “hematopoietic stem and progenitor cell” and “HSPC” refer to a hematopoietic stem cell (HSC), a hematopoietic progenitor cell (HPC), or a population of hematopoietic stem cells and hematopoietic progenitor cells.

### 4. CRISPR/Cas Systems

The present disclosure is based in part on the identification of CRISPR guide sequences that specifically direct the cleavage of an EPOR gene by RNA-guided nucleases, in particular within coding sequences encoding the EPOR cytoplasmic domain. The present disclosure provides a CRISPR/AAV6-mediated genome editing method that can achieve high rates of targeted integration at the EPOR locus. The present disclosure also provides guide sequences for safe-harbor loci such as CCR5, HBA1, and HBB, for methods in which a EPOR transgene is integrated into an HSPC genome outside of the EPOR locus.

Because of the dominant nature of tEPOR, cleavage by the RNA-guided nuclease at the sgRNA target site can occur at one or both copies of a target locus in a cell. In some embodiments, the cleavage of an EPOR locus will lead to an indel that will result in the expression of tEPOR in the cell, i.e., under the control of the endogenous EPOR promoter. In some embodiments, cleavage of an EPOR locus in the presence of a donor template leads to integration of a EPOR transgene at the EPOR locus, and consequently to the expression of tEPOR in the cell under the control of the endogenous EPOR promoter. In some embodiments, cleavage of a target sequence in a safe-harbor locus such as CCR5, HBA1, or HBB in the presence of a donor template leads to integration of a tEPOR transgene and optionally a therapeutic transgene encoding a protein at the safe harbor locus. In such embodiments, the tEPOR cDNA and/or therapeutic transgene can be under the control of a heterologous promoter such as PGK1 or UBC. In some embodiments, the integrated tEPOR cDNA and/or therapeutic transgene is under the control of the endogenous promoter of the safe-harbor locus, e.g., the CR5, HBA1, or HBB promoter.

sgRNAs

In some embodiments, the single guide RNAs (sgRNAs) of the present disclosure target the EPOR locus, in particular within a cytoplasmic-domain coding region such as exon 7 or 8. In some embodiments, the sgRNAs target a safe-harbor locus such as CCR5. HBA1, or HBB, sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at, e.g., an EPOR, HBA1, HBB, or CCR5 locus, and a constant region that mediates binding to Cas9 or another RNA-guided nuclease. The sgRNA can target any sequence within the target gene adjacent to a PAM sequence. The targeted sequence can be within a coding sequence or a non-coding sequence of the gene. In some embodiments, the target sequence comprises one of the sequences shown as SEQ ID NOS:11-14 and 21-23, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, one of SEQ ID NOS: 11-14 and 21-23. In particular embodiments, the target sequence comprises the sequence of SEQ ID NO:11 or SEQ ID NO:12, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to, SEQ ID NO:11 or SEQ ID NO:12. In embodiments wherein an HBA1 or CCR5 safe-harbor locus is targeted, the guide RNA target sequence comprises the sequence of SEQ ID NO: 13 or SEQ ID NO:14, respectively, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to, SEQ ID NO:13 or SEQ ID NO:14. In embodiments wherein an HBB safe-harbor locus is targeted, the guide RNA target sequence comprises the sequence of any one of SEQ ID NOS:21-23, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to, any one of SEQ ID NOS:21-23.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g., 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′4)-methyl-3′-phosphoacetate modifications, 2′-fluoro-pyrimidines. S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific target (e.g., EPOR. CCR5, HBA1, or HBB) sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at, e.g., the EPOR, CCR5, HBA1, or HBB locus. An exemplary CRISPR/Cas system comprises (a) a Cas (e.g., Cas9) or Cpf1 polypeptide or a nucleic acid encoding said polypeptide, and (b) an sgRNA that hybridizes specifically to EPOR, (or CCR5, HBA1, HBB, or other safe-harbor locus), or a nucleic acid encoding said guide RNA. In some instances, the nuclease systems described herein further comprise a donor template as described herein. In particular embodiments, the CRISPR/Cas system comprises an RNP comprising an sgRNA targeting EPOR (or CCR5, HBA1, or HBB) and a Cas protein such as Cas9. In some embodiments, the Cas9 is a high fidelity (HiFi) Cas9 (see, e.g., Vakulskas, C. A. et al., Nat. Med. 24, 1216-1224 (2018)).

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the EPOR locus (or, e.g., the CCR5. HBA1, or HBB locus) to carry out the methods disclosed herein.

Introducing the sgRNA and Cas Protein into Cells

The guide RNA and nuclease can be introduced into the cell using any suitable method. e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the guide RNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP). As used herein, an RNP for use in the present methods can comprise any of the herein-described guide RNAs and any of the herein-described RNA-guided nucleases.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with a genetic condition involving erythroid cells (e.g., α-thalassemia, βthalassemia, sickle cell disease), or from a subject with a condition that could be treated with genetically modified HSPCs expressing a beneficial and/or therapeutic protein (e.g., hemophilia B, phenylketonuria, mucopolysaccharidosis type 1, Gaucher disease, Krabbe disease). In some embodiments, a method is provided of treating a subject with any of the herein-described conditions or disorders (e.g., α-thalassemia, βthalassemia, sickle cell disease, hemophilia B, phenylketonuria, Gaucher disease, Krabbe disease) comprising genetically modifying a plurality of HSPCs isolated from the subject so as to integrate a therapeutic transgene for the particular condition or disorder (e.g., a transgene encoding α-globin, β-globin, factor IX, phenylalanine hydroxylase (PAH), iduronidase, glucocerebrosidase, galactocerebrosidase, and the like), and also to effect the expression of tEPOR as described herein, and reintroducing the HSPCs into the subject. In certain embodiments, the therapeutic transgene is a full-length (e.g., from start codon to stop codon, including introns) transgene comprising a corrective (e.g., wild-type) sequence of an endogenous gene containing one or more deleterious mutations in the HSPCs or encoding a protein that is deficient in the HSPCs. In particular embodiments, HSPCs expressing tEPOR and comprising a therapeutic transgene or other beneficial genetic modification proliferate more rapidly in vivo and become enriched relative to equivalent HSPCs not expressing a tEPOR, e.g., in cells from the subject that have not been genetically modified using the present methods.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain a tEPOR transgene integrated into the EPOR (or CCR5, HBA1, or HBB) locus, and/or cells that have been modified to express a therapeutic or otherwise beneficial transgene. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at the EPOR (or CCR5, HBA1, or HBB) locus, and optionally (b) a homologous donor template or vector as described herein. Each component can be introduced into the cell directly or can be expressed in the cell by introducing a nucleic acid encoding the components of said one or more nuclease systems.

In some aspects, the present methods target integration of a truncated EPOR transgene, i.e., tEPOR, at the endogenous EPOR locus or at a safe harbor locus such as CCR5, HBA1, or HBB in a host cell ex vivo. In some embodiments, the present methods can comprise (a) introducing a donor template comprising a therapeutic transgene encoding a protein and the tEPOR transgene at a safe harbor locus in the genome of the cell, e.g., to introduce a therapeutic genetic modification (such as the introduction of an HBA1, HBA2, HBB, PDGFB, FIX, LDLR, PAH, IDUA, GBA, or GALC transgene or vector into the cell at the safe harbor locus), optionally after expanding said cells, or optionally before expanding said cells, and (b) optionally culturing the cell. In particular embodiments, the first and second homology regions of the donor template flank both the therapeutic transgene and the tEPOR transgene. In certain embodiments, the donor template is a bicistronic cassette comprising an internal ribosome entry site (IRES) between the therapeutic transgene and the tEPOR transgene. An exemplary IRES sequence is shown as SEQ ID NO:24. In certain other embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the therapeutic transgene and the tEPOR transgene. Exemplary nucleic acid sequences encoding T2A and P2A cleavage peptides are shown as SEQ ID NOS:25 and 26, respectively. In some instances, the 2A cleavage peptide is a T2A or P2A cleavage peptide. In other instances, the 2A cleavage peptide is a peptide having sequence similarity and functional interchangeability to a T2A or P2A cleavage peptide, such as an E2A or F2A cleavage peptide. In some instances, the therapeutic transgene is 5′ of the IRES sequence or the sequence encoding the 2A cleavage peptide and the tEPOR transgene is 3′ of the IRES sequence or the sequence encoding the 2A cleavage peptide. In some such instances, the first homology region is 5′ of the therapeutic transgene and the second homology region is 3′ of the tEPOR transgene. In other instances, the tEPOR transgene is 5′ of the IRES sequence or the sequence encoding the 2A cleavage peptide and the therapeutic transgene is 3′ of the IRES sequence or the sequence encoding the 2A cleavage peptide. In some such instances, the first homology region is 5′ of the tEPOR transgene and the second homology region is 3′ of the therapeutic transgene. In other embodiments, the present methods can further comprise (a) introducing a second guide RNA and donor template into the cell, e.g., to introduce a second, therapeutic genetic modification (such as the introduction of an HBA1, HBA2, HBB, PDGFB. FIX, LDLR, PAH, IDUA, GBA, or GALC transgene or vector into the cell at a second genomic locus), optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA as described herein, and optionally (b) a homologous donor template or vector as described herein.

In any of these methods, the nuclease can produce one or more single stranded breaks within the targeted (e.g., EPOR. CCR5, HBA1, or HBB) locus, or a double-stranded break within the targeted locus. In these methods, the targeted locus is modified by homologous recombination with a donor template or vector to result in insertion of the transgene into the locus. The methods can further comprise (c) selecting cells that contain the integrated transgene at the targeted locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 can be introduced into the cell, e.g., by electroporation at the same time as an sgRNA-Cas9 RNP. The sequence of i53 can be found, inter alia, at www.addgene.org/92170/sequences/.

Techniques for the insertion of transgenes, including large transgenes, capable of expressing functional proteins, including enzymes, cytokines, antibodies, and cell surface receptors are known in the art (See, e.g., Bak and Porteus. Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR): O'Connell et al., 2010 PLoS ONE 5(8): e12009 (expression of human IL-7); Tuszynski et al., Nat Med. 2005 May; 11(5):551-5 (expression of NGF in fibroblasts): Sessa et al., Lancet. 2016 Jul. 30:388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin): Bak and Porteus, Cell Reports. Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The transgene to be integrated, which is comprised by a polynucleotide or donor construct, can be any tEPOR transgene whose gene product can provide tEPOR expression in red blood cells or other cells of the erythroid lineage, and particularly provide EPO hypersensitivity and/or drive the elevated proliferation of the modified cells relative to equivalent cells lacking the tEPOR. For example, the transgene could be used to modify an endogenous EPOR gene, e.g., convert a wild type EPOR allele into a tEPOR-encoding allele. In some embodiments, a tEPOR transgene is integrated at a genomic location outside of the EPOR locus. For example, in some embodiments, a tEPOR transgene and optionally a therapeutic transgene encoding a protein is integrated at a safe harbor locus such as CCR5, HBA1, or HBB. In particular embodiments, the tEPOR transgene in the homologous repair template comprises SEQ ID NO:10 or a subsequence of SEQ ID NO:10, or a sequence comprising at least about 70%, 75%, 80%, 85%, 90%, 95%, 96%, 97%, 98%, 99%, or more identity to SEQ ID NO:10 or a subsequence of SEQ ID NO:10. In some embodiments, the tEPOR transgene comprises a nonsense mutation in a location within the gene encoding the cytoplasmic domain. In some embodiments, the tEPOR transgene comprises a nonsense mutation in exon 7 or exon 8 of the full length EPOR. In some embodiments, the tEPOR transgene comprises a G to A transition in nucleotide 6002 of the EPOR gene that converts a TGG codon for tryptophan into a TAG stop codon, causing the truncation of the 70 C-terminal amino acids of the EPOR molecule. In some embodiments, the EPOR transgene encodes a tEPOR lacking the C-terminal 70 amino acids of a full-length EPOR, or lacking 65, 66, 67, 68, 69, 71, 72, 73, 74, or 75 C-terminal amino acids of a full length-EPOR. In some embodiments, the TEPOR transgene encodes a tEPOR lacking an SHP-1 (or SHPTP-1) binding domain of full-length EPOR. In some embodiments, the EPOR transgene encodes a tEPOR lacking about 10, 20, 30, 40, 50, 60, 70, 80, 90, 100, 110, 120, 130, 140, 150, 160, 170, 180, 190, 200, 210, 220, 230, 10-236, 10-50, 50-60, 60-70, 65-75, 70-80, 80-90, 90-100, 100-150, 150-200, or 200-236 C-terminal amino acids of a full-length EPOR. In some embodiments, the donor template comprises the sequence shown as SEQ ID NO:3 or a subsequence thereof (e.g., a subsequence lacking a GFP marker gene), or a sequence comprising at least about 70%, 75%, 80%, 85%, 90%, 95%, 96%, 97%, 98%, 99%, or more identity to SEQ ID NO:3 or a subsequence thereof (e.g., a subsequence lacking a GFP marker gene).

In some embodiments, the donor template further comprises a therapeutic transgene. An exemplary, non-limiting list of suitable transgenes includes HBA1 (hemoglobin subunit alpha 1; see, e.g., NCBI Gene ID No. 3039), HBA2 (hemoglobin subunit alpha 2; see, e.g., NCBI Gene ID No. 3040), HBB (hemoglobin subunit beta; see, e.g., NCBI Gene ID No. 3043), PDFGB (platelet-derived growth factor subunit B; see, e.g., NCBI Gene ID No. 5155), IDUA (alpha-L-iduronidase; see, e.g., NCBI Gene ID No. 3425), PAH (phenylalanine hydroxylase; see, e.g., NCBI Gene ID No. 5053), Factor IX (or FIX; see, e.g., NCBI Gene ID NO. 2158), including Hyperactive Factor IX Padua, or the Padua Variant (see. e.g., Simioni et al., (2009) NEJM 361:1671-1675; Cantore et al. (2012) Blood 120:4517-4520; Monahan et al., (2015) Hum. Gene. Ther. 26:69-81), LDLR (low density lipoprotein receptor; see, e.g., NCBI Gene ID No. 3949), and others. In particular embodiments, the first and second homology arms of the donor template flank both the therapeutic transgene and the tEPOR transgene. In certain embodiments, the donor template is a bicistronic cassette comprising an internal ribosome entry site (IRES) between the therapeutic transgene and the tEPOR transgene. In certain other embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the therapeutic transgene and the tEPOR transgene. In some instances, the therapeutic transgene is 5′ of the IRES sequence or the sequence encoding the 2A cleavage peptide and the tEPOR transgene is 3′ of the IRES sequence or the sequence encoding the 2A cleavage peptide. In some such instances, the first homology arm is 5′ of the therapeutic transgene and the second homology arm is Y of the tEPOR transgene. In other instances, the tEPOR transgene is 5′ of the IRES sequence or the sequence encoding the 2A cleavage peptide and the therapeutic transgene is 3′ of the IRES sequence or the sequence encoding the 2A cleavage peptide. In some such instances, the first homology arm is 5′ of the tEPOR transgene and the second homology arm is 3′ of the therapeutic transgene.

In other embodiments, a second donor template is used that comprises a therapeutic transgene, e.g., an HBA1, HBA2, HBB, PDGFB, IDUA, GBA, FIX, LDLR, PAH, or GALC transgene. In some embodiments, the second donor template comprises a sequence shown as SEQ ID NO:6 or SEQ ID NO:9 or a subsequence thereof (e.g., a subsequence lacking the YFP marker gene), or a sequence comprising at least about 70%, 75%, 80%, 85%, 90%, 95%, 96%, 97%, 98%, 99%, or more identity to SEQ ID NO:6 or a subsequence thereof or SEQ ID NO:9 or a subsequence thereof (e.g., a subsequence lacking the YFP marker gene).

In some embodiments, expression of a tEPOR transgene is driven by an endogenous EPOR promoter, e.g., in embodiments where the transgene is integrated at an EPOR locus. In some embodiments, a tEPOR transgene, and/or a second transgene such as an HBA1, HBA2, HBB, IDUA, PDGFB, GBA, FX. LDLR, PAH, or GALC transgene, is driven by a heterologous promoter such as HBA1, HBA2, HBB, PGK1, or UBC. In other embodiments, a tEPOR transgene, and/or a second transgene such as an HBA1, HBA2, HBB, IDUA, PDGFB, GBA, FIX, LDLR, PAH, or GALC transgene, is driven by an endogenous promoter such as HBA1, HBA2, or HBB.

In some embodiments, the transgene in the homologous repair template is codon-optimized, e.g., comprises at least about 70%, 75%, 80%, 85%, 90%, 95%, or more homology to the corresponding wild-type coding sequence or cDNA, or a fragment thereof such as in the case of a truncated EPOR.

A transgene as used herein may also comprise optional elements such as introns, WPREs, polyA regions, UTRs (e.g., 5′ or 3′ UTRs). In particular embodiments, the template comprises a polyA sequence or signal. e.g., a bovine growth hormone polyA sequence or a rabbit beta-globin polyA sequence, at the 3 end of the cDNA. In particular embodiments, a Woodchuck Hepatitis Virus Posttranscriptional Regulatory Element (WPRE) is included within the 3′UTR of the template, e.g., between the 3′ end of the coding sequence and the 5′ end of the polyA sequence, so as to increase the expression of the transgene. Any suitable WPRE sequence can be used: See, e.g., Zufferey et al. (1999) J. Virol. 73(4):2886-2892: Donello, et al. (1998). J Virol 72: 5085-5092; Loeb, et al. (1999). Hum Gene Ther 10: 2295-2305: the entire disclosures of which are herein incorporated by reference).

To facilitate homologous recombination, the transgene is flanked within the polynucleotide or donor construct by sequences homologous to the target genomic sequence. In particular embodiments, the transgene is flanked by one sequence homologous to the region 5′ to the cleavage site (e.g., starting at or around the guide RNA target sequence and running upstream) and a second sequence homologous to the region 3′ of the site of cleavage (e.g., starting at or around the guide RNA target site and running downstream). In some embodiments, one of the homologous regions (e.g., the region 5′ to the cleavage site) comprises sequences upstream of the translation start site. In particular embodiments, the EPOR left homology arm comprises the sequence shown as SEQ ID NO:1 or a subsequence thereof, or to a sequence comprising at least about 70%, 75%, 80%, 85%, 90%, 95%, 96%, 97%, 98%, 99% or more homology to SEQ ID NO:1 or a subsequence thereof. In particular embodiments, the EPOR right homology arm comprises the sequence shown as SEQ ID NO:2 or a subsequence thereof, or to a sequence comprising at least about 70%, 75%, 80%, 85%, 90%, 95%, 96%, 97%, 98%, 99% or more homology to SEQ ID NO:2 or a subsequence thereof. In some embodiments wherein the HBA1 safe harbor locus is targeted, the donor template comprises a left homology sequence comprising the sequence shown as SEQ ID NO:4 or a subsequence thereof, or to a sequence comprising at least about 70%, 75%, 80%, 85%, 90%, 95%, 96%, 97%, 98%, 99% or more homology to SEQ ID NO:4 or a subsequence thereof. In some embodiments wherein the HBA1 safe harbor locus is targeted, the donor template comprises a right homology sequence comprising the sequence shown as SEQ ID NO:5 or a subsequence thereof, or to a sequence comprising at least about 70%, 75%, 80%, 85%, 90%, 95%, 96%, 97%, 98%, 99% or more homology to SEQ ID NO:5 or a subsequence thereof. In some embodiments wherein the CCR5 safe harbor locus is targeted, the donor template comprises a left homology sequence comprising the sequence shown as SEQ ID NO:7 or a subsequence thereof, or to a sequence comprising at least about 70%, 75%, 80%, 85%, 90%, 95%, 96%, 97%, 98%, 99% or more homology to SEQ ID NO:7 or a subsequence thereof. In some embodiments wherein the CCR5 safe harbor locus is targeted, the donor template comprises a right homology sequence comprising the sequence shown as SEQ ID NO:8 or a subsequence thereof, or to a sequence comprising at least about 70%, 75%, 80%, 85%, 90%, 95%, 96%, 97%, 98%, 99% or more homology to SEQ ID NO:8 or a subsequence thereof. In some embodiments wherein the HBB safe harbor locus is targeted, the donor template comprises a left homology sequence comprising the sequence shown as SEQ ID NO:19 or a subsequence thereof, or to a sequence comprising at least about 70%, 75%, 80%, 85%, 90%, 95%, 96%, 97%, 98%, 99% or more homology to SEQ ID NO:19 or a subsequence thereof. In some embodiments wherein the HBB safe harbor locus is targeted, the donor template comprises a right homology sequence comprising the sequence shown as SEQ ID NO:20 or a subsequence thereof, or to a sequence comprising at least about 70%, 75%, 80%, 85%, 90%, 95%, 96%, 97%, 98%, 99% or more homology to SEQ ID NO:20 or a subsequence thereof.

In some embodiments, a tEPOR transgene replaces all or part of an endogenous coding sequence of EPOR such that its expression is driven by the endogenous EPOR promoter. In some embodiments, a tEPOR transgene (and optionally a therapeutic transgene such as HBA1, HBA2, HBB, PDGFB, FX, LDLR PAH, IDUA, GBA, or GALC) replaces all or part of a safe harbor gene such as CCR5, HBA1, or HBB such that its expression is driven by the endogenous CCR5, HBA1, or HBB promoter. In some embodiments, a tEPOR transgene (and optionally a therapeutic transgene such as HBA1, HBA2, HBB, PDGFB, FIX, LDLR, PAH. IDUA, GBA, or GALC) is integrated into a safe harbor locus such as CCR5. HBA1, or HBB wherein the expression of the transgene is driven by a heterologous promoter such as HBA1, HBA2, HBB, PGK1, or UBC. In some embodiments, a part or a fragment of the target gene is replaced by the transgene. In some embodiments, the whole coding sequence of the target gene is replaced by the transgene. In some embodiments, the coding sequence and regulatory sequences of the transgene is replaced by the transgene. In some embodiments, the target gene sequence replaced by the transgene comprises an open reading frame. In some embodiments, the target gene sequence replaced by the transgene comprises an expression cassette. In some embodiments, the target gene sequence replaced by the transgene comprises a sequence that transcribes into a precursor mRNA. In some embodiments, the target gene sequence replaced by the transgene comprises a 5′ UTR, one or more introns, one or more exons, and a 3′ UTR.

In some embodiments, the 5′ (or left) homology arm is at least 100 bp, 200 bp, 300 bp, 400 bp, 500 bp, 600 bp, 700 bp, 800 bp, 900 bp, 1000 bp or more in length. In some embodiments, the 5′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 5′ homology arm is at least 400 bp in length. In some embodiments, the 5′ homology arm is at least 500 bp, 600 bp, 700 bp, 800 bp, 900 bp, or 1000 bp in length. In some embodiments, the 5′ homology arm is at least 850 bp in length. In some embodiments, the 5′ homology arm is 400-500 bp. In some embodiments, the 5′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, I100-1200 bp, 1200-1300 bp, 1300-1400 bp, or 1400-1500 bp in length. In particular embodiments, the 5′ homology arm is about 900 nucleotides in length.

In some embodiments, the 3′ (or right) homology arm is at least 100 bp, 200 bp, 300 bp, 400 bp, 500 bp, 600 bp, 700 bp, 800 bp, 900 bp, 1000 bp or more in length. In some embodiments, the 3′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 3′ homology arm is at least 400 bp in length. In some embodiments, the 3′ homology arm is at least 500 bp, 600 bp, 700 bp, 800 bp, 900 bp, or 1000 bp in length. In some embodiments, the 3′ homology arm is at least 850 bp in length. In some embodiments, the 3′ homology arm is 400-500 bp. In some embodiments, the 3′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-11100 bp, 1100-1200 bp, 1200-1300 bp, 1300-1400 bp, or 1400-1500 bp in length. In particular embodiments, the 3′ homology arm is about 900 nucleotides in length.

Any suitable method can be used to introduce the polynucleotide, or donor construct, into the cell. In particular embodiments, the polynucleotide is introduced using a recombinant adeno-associated viral vector (rAAV). For example, the rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In particular embodiments, the vector is an rAAV6 vector. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the EPOR locus, and (b) a tEPOR transgene as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g., an AAV backbone, to generate virus; (2) arms of homology to the target site of at least 200 bp but ideally at least 400 bp or at least 900 on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a functional alpha globin protein and capable of expressing the functional alpha globin protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

The inserted construct can also include other safety switches, such as a standard suicide gene into the locus (e.g., iCasp9) in circumstances where rapid removal of cells might be required due to acute toxicity. The present disclosure provides a robust safety switch so that any engineered cell transplanted into a body can be eliminated. e.g., by removal of an auxotrophic factor. This is especially important if the engineered cell has transformed into a cancerous cell.

The present methods allow for the efficient integration of the tEPOR donor template at the endogenous EPOR locus. In some embodiments, the present methods allow for the insertion of the donor template in at least about 5%, 10%, 15%, 20%, 25%, 30%, 35%, 40%, 45%, 50%, 55%, 60%, 65%, 70%, 75%, 80%, 85%, 90%, 95%, or more cells, e.g., HSPCs from a subject.

In some embodiments, the CRISPR-mediated systems as described herein (e.g., comprising a guide RNA, RNA-guided nuclease, and optionally homologous repair template) are assessed in primary HSPCs, e.g., as derived from mobilized peripheral blood or from cord blood. In such embodiments, the HSPCs can be WT primary HSPCs (e.g., for initial testing of the system) or from patient-derived HSPCs (e.g., for pre-clinical in vitro testing). In some embodiments, the HSPCs are cultured in vitro and allowed to differentiate into RBCs to confirm the elevated rate of proliferation relative to unmodified cells (as measured, e.g., by a co-culture experiment in the presence or absence of EPO as described in Example 1 or by other methods of determining proliferation rate such as BrdU incorporation or by monitoring the number of cells in a culture over time) prior to the reintroduction of HSPCs into a subject.

### 5. Methods of Treatment

Following the integration of the tEPOR transgene into the genome of the HSPC and, in particular embodiments, of a therapeutic transgene as described herein, and optionally following confirmation of expression of the encoded therapeutic protein and/or of the elevated proliferation rate of the cells, a plurality of modified HSPCs can be reintroduced into the subject. In one embodiment, the HSPCs are introduced by intrafemoral injection, such that they can populate the bone marrow and differentiate into, e.g., red blood cells. In some embodiments, the HSPCs are introduced by intravenous injection. In some embodiments, the HSPCs are induced to initiate differentiation into red blood cells in vitro, and the modified erythroid lineage cells are then re-introduced into the subject.

Disclosed herein, in some embodiments, are methods of treating a genetic condition or disorder (e.g., α-thalassemia. β-thalassemia, sickle cell disease, hemophilia B, phenylketonuria, mucopolysaccharidosis type 1, Gaucher disease. Krabbe disease, and the like) in an individual in need thereof, the method comprising genetically modifying HSPCs from the individual so as to provide a beneficial effect (e.g., by introducing a therapeutic transgene for correcting a mutation underlying the condition or disorder, or for providing to the individual a protein replacement therapy) and also such that they express tEPOR and are therefore enriched in vivo following reintroduction of the cells to the individual.

The present methods allow for the efficient integration of a donor template comprising a therapeutic transgene and a tEPOR transgene at a safe harbor locus. In some embodiments, expression of the therapeutic transgene and the tEPOR transgene causes an enrichment of genetically modified HSPCs in a population of HSPCs, e.g., over the course of red blood cell differentiation, as compared to expression of the therapeutic transgene in the absence of expression of the tEPOR transgene. In certain embodiments, the present methods allow for the insertion of the donor template in at least about 5%, 10%, 15%, 20%, 25%, 30%, 35%, 40%, 45%, 50%, 55%, 60%, 65%, 70%, 75%, 80%, 85%, 90%, 95%, or more HSPCs in a population of HSPCs, e.g., over the course of red blood cell differentiation. In certain embodiments, expression of the therapeutic transgene and the EPOR transgene increases the proportion of genetically modified HSPCs in a population of HSPCs by at least about 5%, 10%, 15%, 20%, 25%, 30%, 35%, 40%, 45%, 50%, 55%, 60%, 65%, 70%, 75%, 80%, 85%, 90%, 95%, or more, e.g., over the course of red blood cell differentiation, as compared to expression of the therapeutic transgene in the absence of expression of the tEPOR transgene. In some embodiments, expression of the therapeutic transgene and the tEPOR transgene increases a level of adult hemoglobin tetramers in the genetically modified HSPCs by at least about 5%, 10%, 15%, 20%, 25%, 30%, 35%, 40%, 45%, 50%, 55%, 60%, 65%, 70%, 75%, 80%, 85%, 90%, 95%, or more as compared to expression of the therapeutic transgene in the absence of expression of the tEPOR transgene.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell as described herein is provided. The modified autologous host cell is genetically engineered to comprise an integrated tEPOR transgene at the EPOR locus or at a safe harbor locus such as CCR5, HBA1, or HBB, as well as optionally a second, therapeutic genetic modification as described herein (e.g., a therapeutic transgene integrated at the safe harbor locus). The modified host cell of the disclosure herein may be formulated using one or more excipients to, e.g.: (1) increase stability: (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical.

In some embodiments, a subject undergoes a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007): Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the condition or disorder, e.g., α-thalassemia, β-thalassemia, sickle cell disease, etc. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the condition or disorder being treated and the severity of the condition or disorder; the activity of the specific payload employed; the specific composition employed; the age, body weight, general health, sex and diet of the patient; the time of administration, route of administration; the duration of the treatment: drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of α-thalassemia, β-thalassemia, sickle cell disease, hemophilia B, phenylketonuria, mucopolysaccharidosis type 1, Gaucher disease, Krabbe disease or other genetic conditions or disorders is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. EXAMPLES

The present disclosure will be described in greater detail byway of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Using C-Terminal Truncated Forms of EPOR to Enrich HSCs Differentiating Down the Erythroid Lineage

The present example describes a method for using truncations of the EPO Receptor (EPOR), e.g., a specific truncation of the remaining ˜70 C-terminal amino acids of the EPO receptor, to safely enrich create edited HSCs that preferentially differentiate down the erythroid lineage. This method can be used to allow a low HSC edited cell chimerism in the bone marrow to yield a high edited RBC chimerism in the bloodstream. Because edited cell chimerism is currently one of the greatest bottlenecks to clinical efficacy of gene therapies for the hemoglobinopathies, this technology has incredible translational potential.

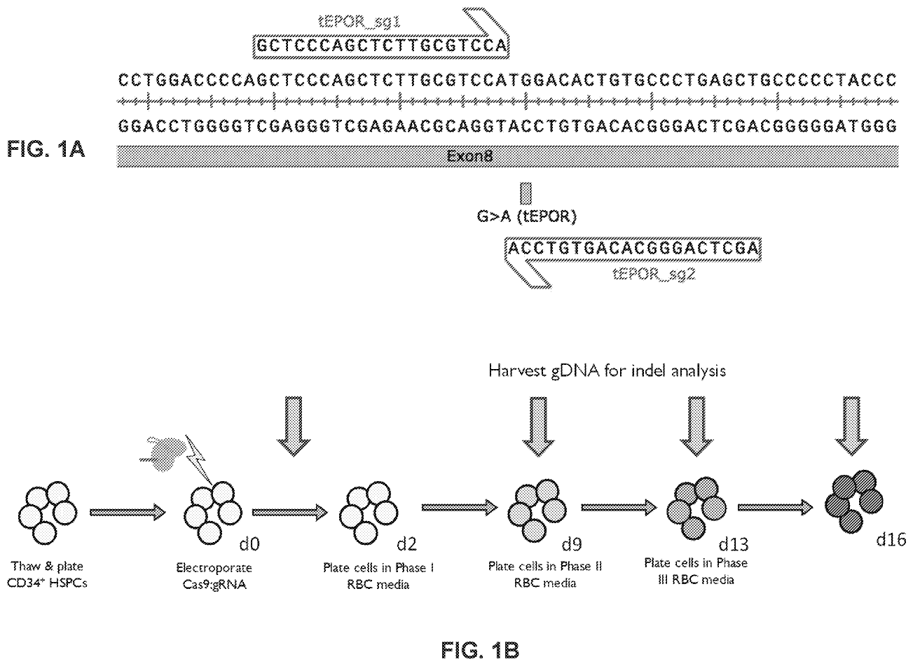

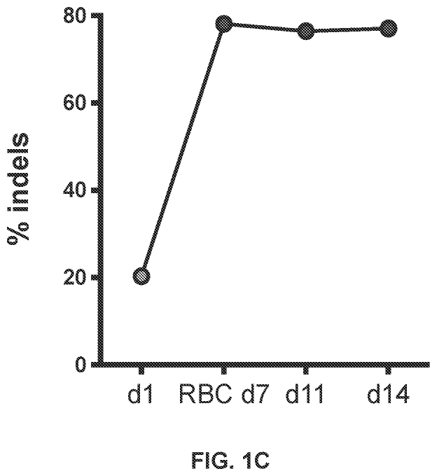

As an initial test, we designed two SpCas9 gRNAs overlapping the variant responsible for the truncation in the Olympic cross-country skier Eero Mäntyranta (FIG. 1A). This was expected to yield an indel spectrum, a subset of which are expected to yield premature truncation of the endogenous EPOR. We delivered these gRNAs pre-complexed with high fidelity variant SpCas9 RNP (Vakulskas, et al. Nature Medicine, 2018) by electroporation to WT HSPCs as previously described (Bak, et al. Nature Protocols, 2018). We then performed a three-phase RBC differentiation protocol and harvested genomic DNA over the course of the 16d protocol to determine whether enrichment of HSPCs with indels occurred (FIG. 1B). The cells were cultured with high EPO levels (20 U/mL). Because the vast majority of editing has occurred by 24-48 h, any enrichment or depletion of edited cells following this is likely to represent a functional consequence of editing. Indeed, we observed ˜4× enrichment of our edited cells occurring rapidly between d1 and d9 which was maintained throughout the remainder of RBC differentiation (FIG. 1C).

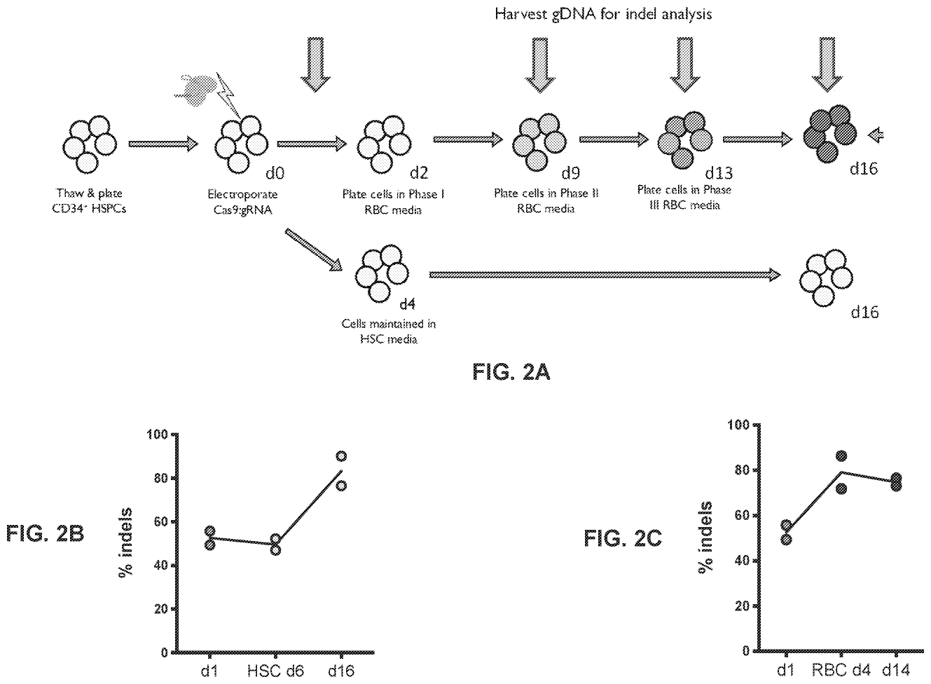

We next tested whether this effect is limited to cells that are erythroid-differentiated or whether enrichment also occurs in edited HSPCs maintained in HSC media (FIG. 2A). We found that enrichment occurred in both conditions, though the effect was more immediate in cells that underwent RBC differentiation (FIG. 2B).

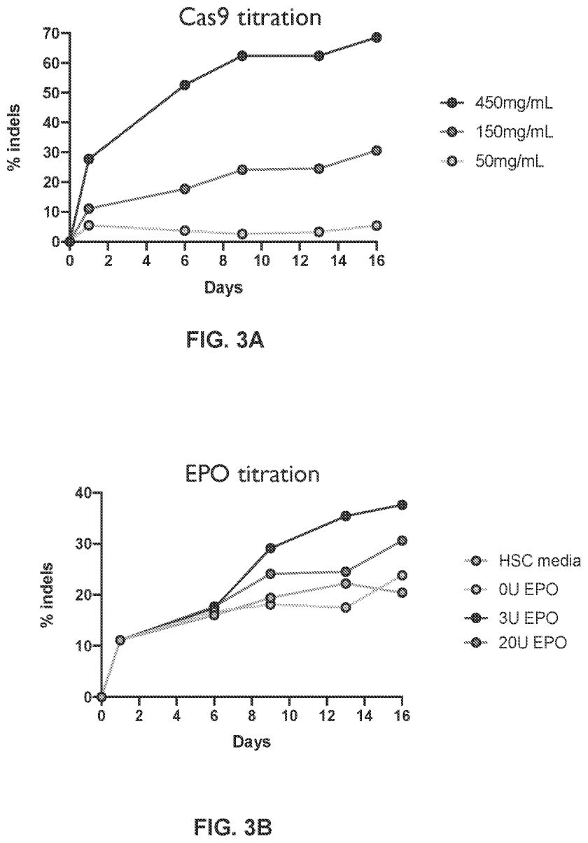

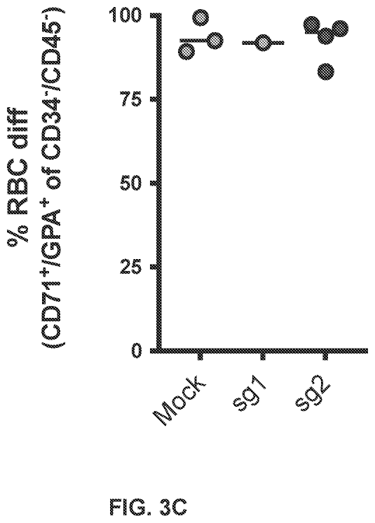

We next titrated the amount of Cas9 and gRNA used in order to determine the degree of enrichment that occurs following lower initial editing frequencies. We found enrichment (from ˜30% to ˜70% indels) in standard conditions (450 mg/mL Cas9) over the course of RBC differentiation which was slightly less pronounced in the ⅓ condition and virtually absent in the 1/9 Cas9 condition (FIG. 3A). FIG. 3B shows the frequency of indels over the course of erythroid differentiation from a single treatment targeted with EPOR sg2, but maintained in either standard HSC media or RBC differentiation media with 0, 3, or 20 U/mL EPO. Note, in normal RBC differentiation, 3 U/mL of EPO is maintained through the 14d protocol. Here, we found that enrichment occurred in all groups, though it was most evident in the 3 and 20 U/mL EPO conditions. As shown in FIG. 3B, we found that editing with these EPOR-truncating gRNAs had no negative impact on the ability of cells to differentiate down the erythroid lineage.

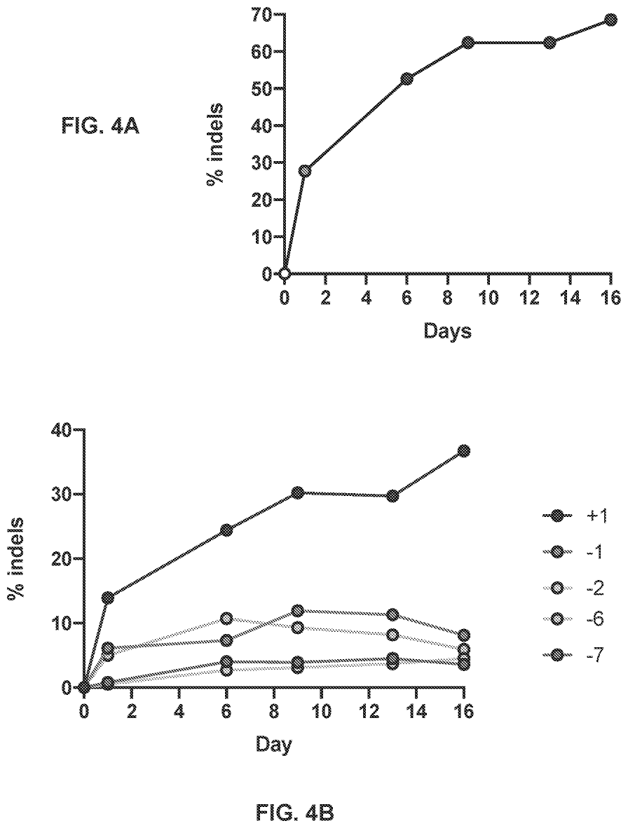

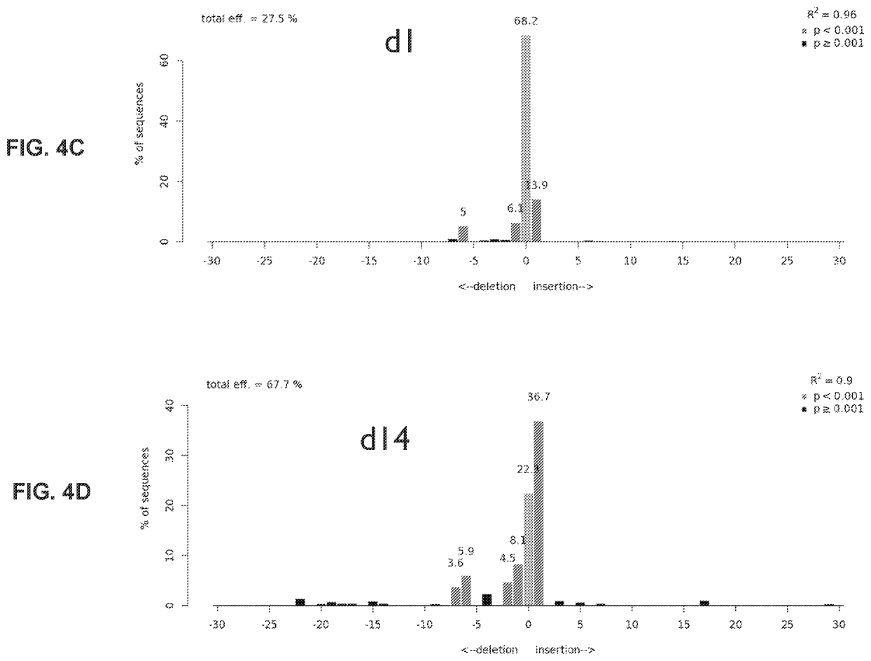

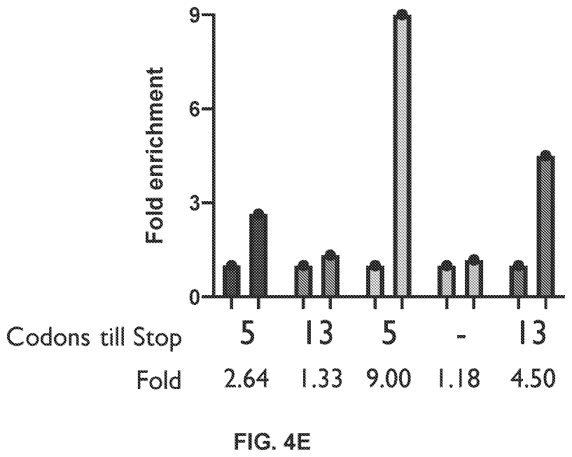

To determine which particular indels are driving the observed enrichment (for this sample, from 27.5% indels at d1 to 67.7% indels at d14; FIG. 4A), we plotted the five most common indels found in this treatment (WT HSPCs targeted with tEPOR sg2 as before) over the course of RBC differentiation (FIGS. 4B-4D). We found that of the five most common indels, the four that introduce downstream truncation events undergo the most enrichment compared to the single indel (a 6 bp deletion) which would maintain the reading frame, and therefore not lead to a truncation (FIG. 4E).

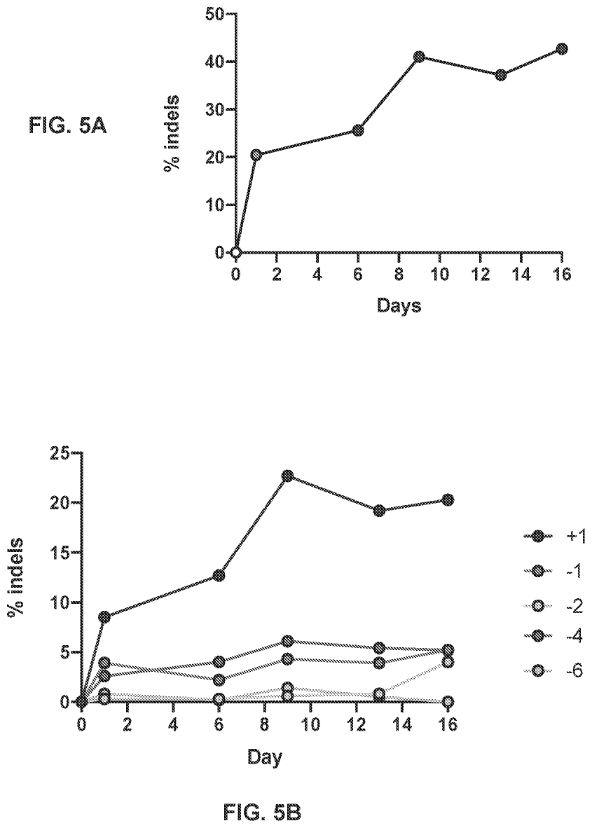

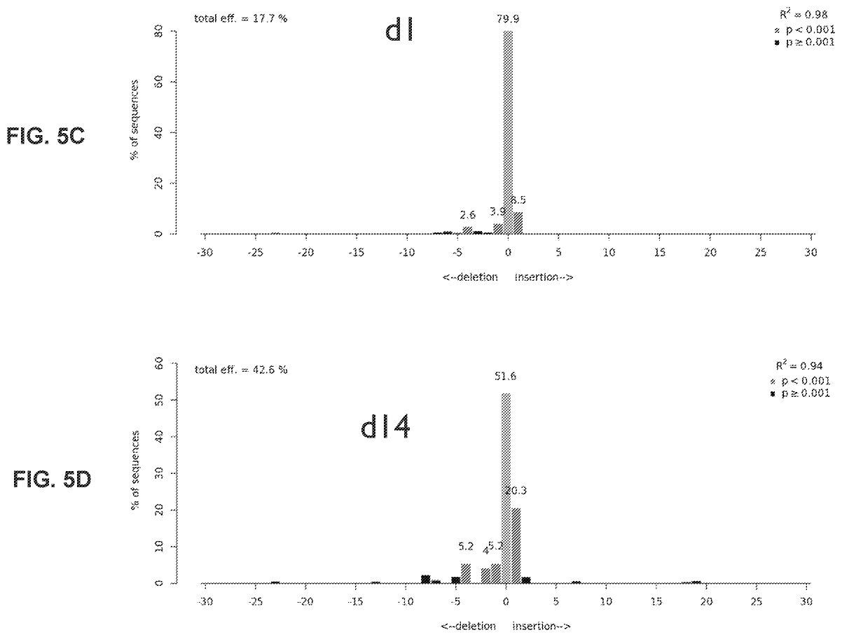

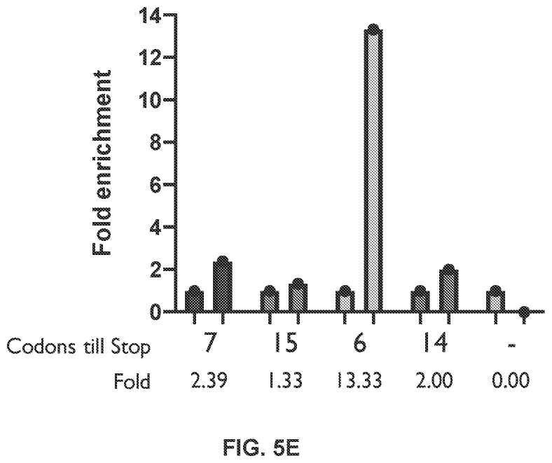

We next plotted similar data as in FIGS. 4A-4E, but for WT HSPCs targeted with tEPOR sg1. Again, of the five most common indels, the four the lead to truncation are enriched (FIG. 5E), while the single indel that maintains the reading frame (a 6 bp deletion) is fully depleted by d14 (FIG. 5D).

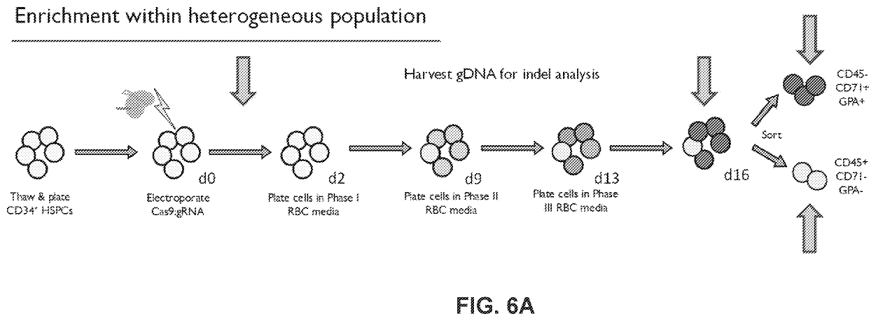

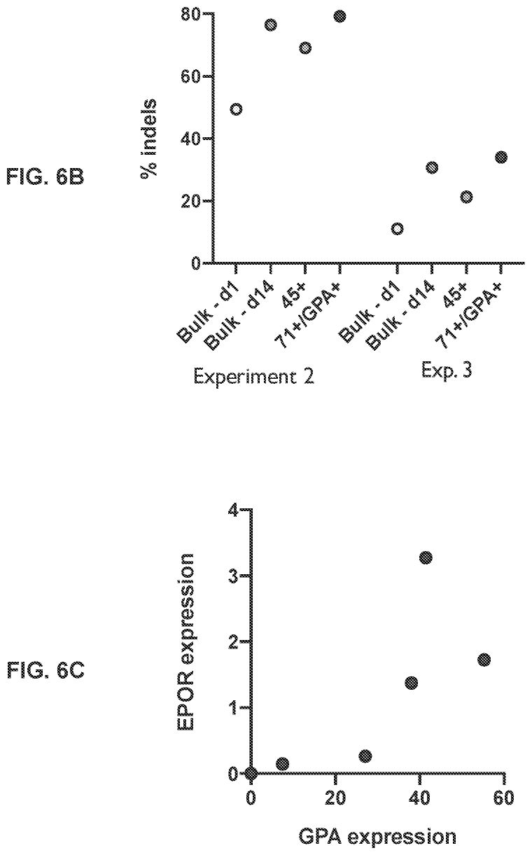

To further investigate the cell types capable of undergoing enrichment, we targeted WT HSPCs as before with tEPOR sg2, performed erythroid differentiation, and then at d16 sorted out cells that both had and had not acquired erythroid markers (CD71 and GPA) (FIG. 6A). We found that while the frequency of indels was enriched in both cell populations, this effect was most pronounced in the cells that had more fully differentiated down the RBC lineage (FIG. 6B). To investigate whether this effect may be driven by EPOR expression, we determined mRNA expression harvested from d16 RBCs by ddPCR and found that EPOR expression directly correlated with GPA expression (FIG. 6C).

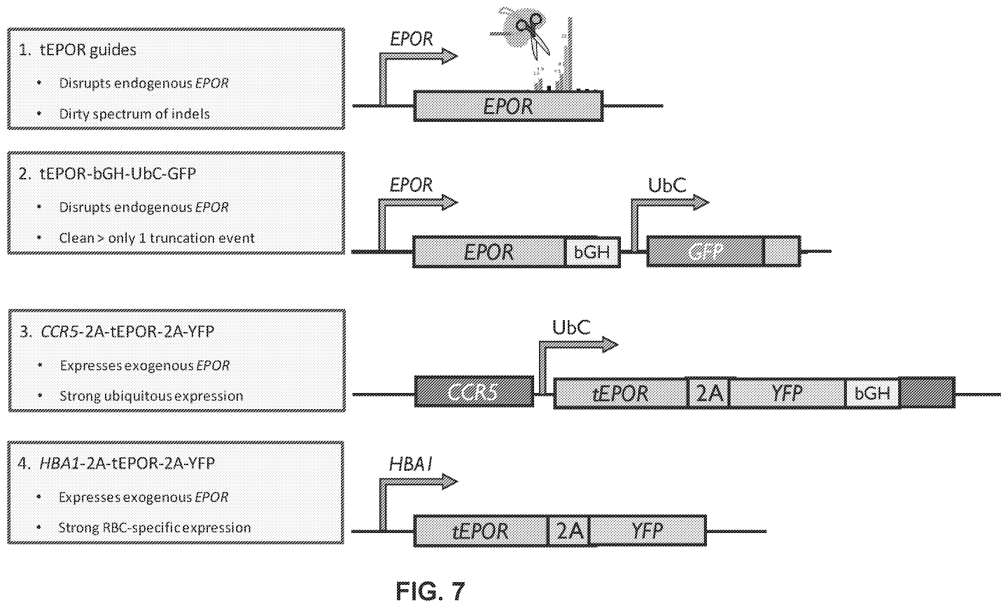

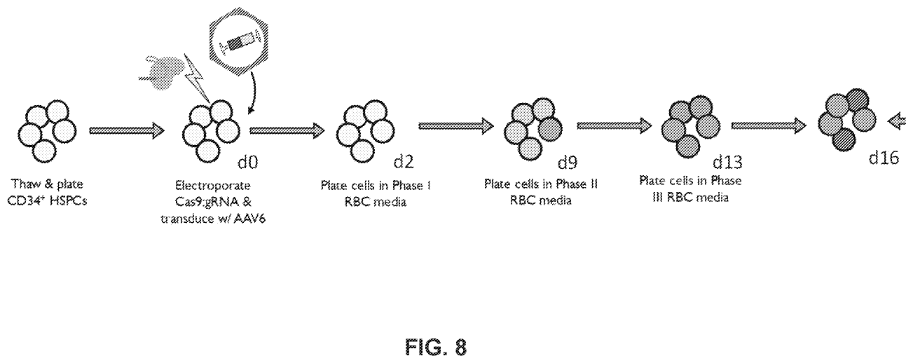

While targeting the region harboring the truncating SNP found in the Olympic cross-country skier (Eero Mäntyranta) with Cas9 gRNAs leads to enrichment, not all indels cause a truncation event. Therefore, we designed a polyA-UbC-GFP integration cassette that could be delivered by AAV6 transduction following cleavage by tEPOR sg2 in order to yield a population of GFP+ cells that all would have at least a heterozygous truncation of EPOR (FIG. 7). This would allow us to measure GFP+ in real-time as cells differentiate into RBCs. In addition to methods 1 and 2 as shown in FIG. 7, which alter the endogenous EPOR (which was found to be relatively lowly expressed), we could alternatively introduce a tEPOR cDNA as a transgene either driven by an endogenous promoter, like the HBA1 erythroid-specific safe harbor site characterized in prior work (Cromer, et al. Nature Medicine, 2021) (FIG. 7). We could also deliver this tEPOR transgene into a typical safe harbor site like CCR5 which would be driven by a custom strong constitutive promoter like UbC (FIG. 7). Because expression in contexts 3 and 4 is likely to produce much greater levels of tEPOR expression compared to disruption of the endogenous gene, the degree of enrichment may be more dramatic in these contexts.

Because constructs 2-4 shown in FIG. 7 all have fluorescent markers that integrate along with tEPOR, we were able to determine enrichment in the various cell populations in real-time over the course of erythroid differentiation using flow cytometry.

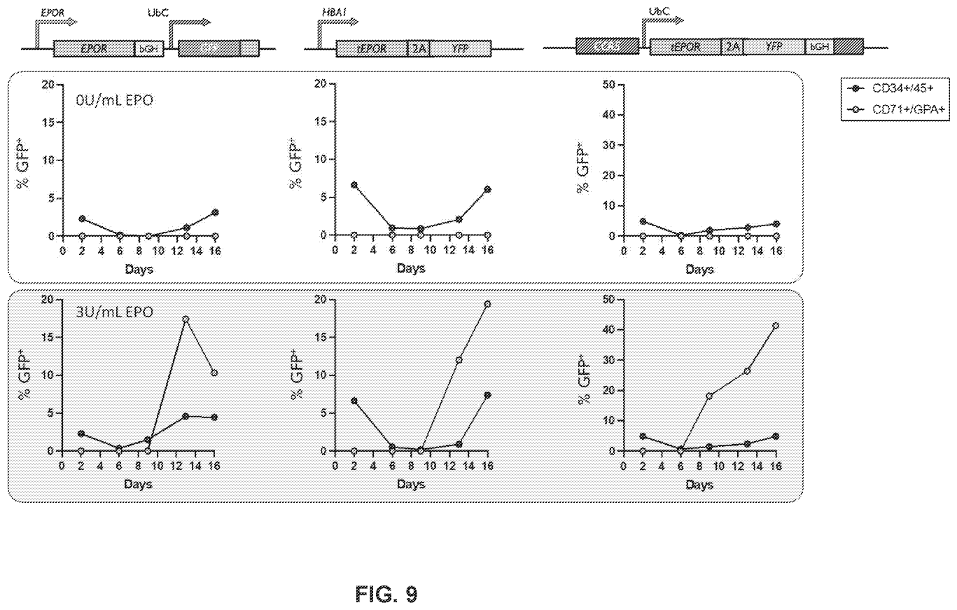

At 2 days post-editing, HSPCs targeted with the constructs shown were plated in RBC differentiation media containing either 0 or 3 U/mL EPO (top row and bottom row of FIG. 9, respectively). The percentage of GFP+ cells was plotted in the more stem-like population (CD34+/CD45+/CD71−/GPA−) as well as the population that acquired RBC-specific markers (CD34−/CD45−/CD71+/GPA+). As expected, the most dramatic enrichment occurred only in the presence of EPO in the RBC population. In spite of strong and/or constitutive expression of tEPOR by the HBA1- and CCR5-integrating constructs, very little enrichment occurred in the absence of EPO or in the stem-like population in the presence of EPO. This indicates that even the tEPOR transgene-integrating constructs retain EPO sensitivity, which indicates they may be a powerful yet safe method of enriching for edited erythroid cells for clinical applications.

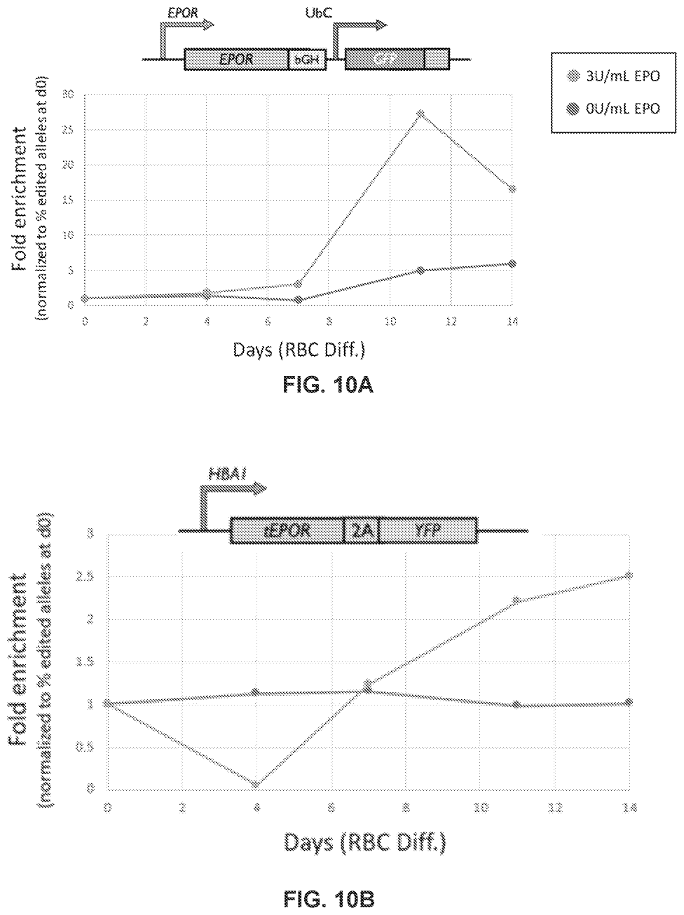

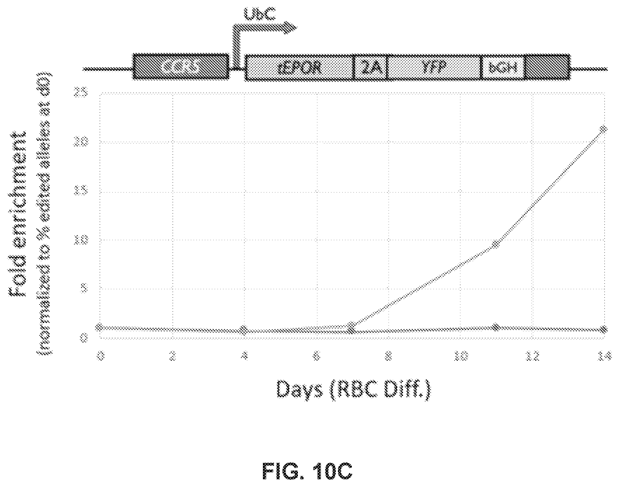

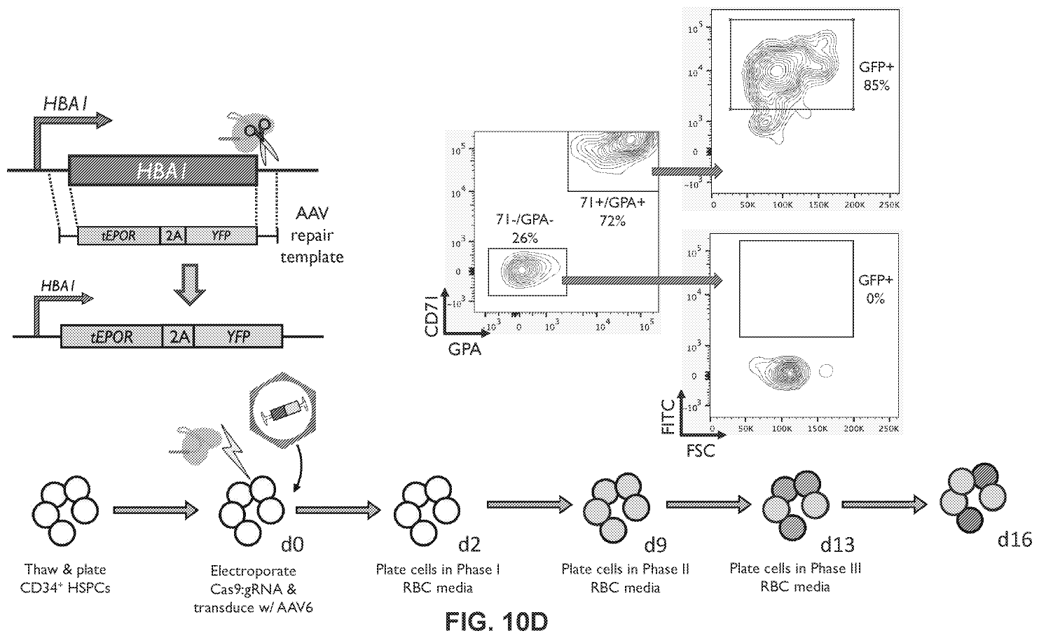

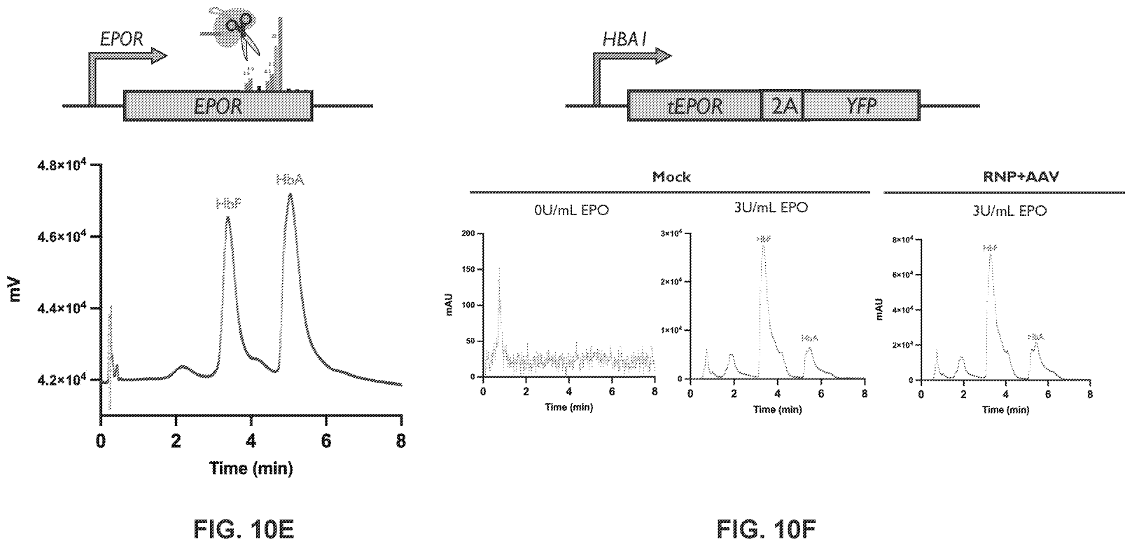

To confirm that editing frequencies did in fact increase over the course of RBC differentiation, ddPCR was used to quantify editing frequencies in the 0 and 3 U/mL EPO conditions depicted in FIG. 9. Indeed, we observed dramatic enrichment of edited cells in all three constructs (FIGS. 10A-10C).

FIG. 10D shows dramatic enrichment when tEPOR is integrated at the HBA1 locus. At d14 of RBC differentiation, we analyzed cells by FACS and observed that YFP was only expressed in cells that had acquired erythroid-specific markers (CD71+/GPA+). In fact, the vast majority of RBCs in this experiment were indeed edited (as indicated by GFP expression). This indicates strong enrichment of RBCs that are expressing tEPOR from the HBA1 locus (a finding that was confirmed at the genomic DNA level by ddPCR, which showed 6× enrichment in bulk cells).

FIGS. 10E-10F show hemoglobin production following tEPOR introduction. We found that cells edited with both the endogenous EPOR-truncating gRNA “sg2” (FIG. 10E) as well as the HBA1-integrating tEPOR cassette (FIG. 10F) are able to yield both fetal and adult hemoglobin (HbF and HbA, respectively). While these two editing strategies were executed on different HSPC donors (which likely accounts for the difference in ratio of HbF to HbA), in the Mock vs. HBA1 (tEPOR), we observed no difference in hemoglobin production in the presence of EPO.

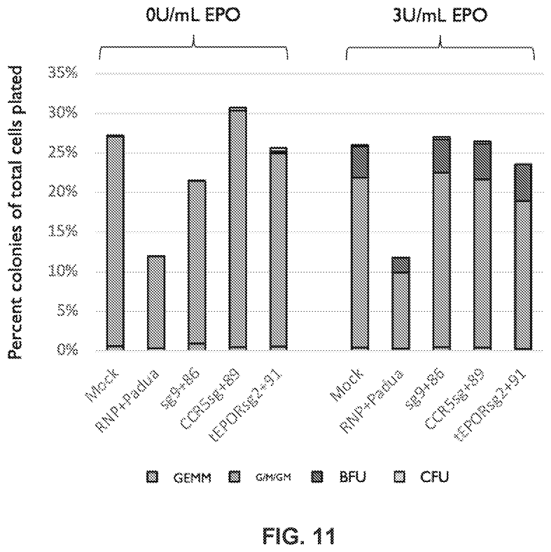

Next, to determine the impact of tEPOR introduction on lineage bias and colony-forming ability of edited HSPCs, we performed CFU assays as previously described (Bak, et al. Nature Protocols, 2016). In the presence or absence of EPO in the methylcellulose, we observed no substantial lineage bias in any of the edited conditions (FIG. 11). Interestingly, while cells edited for a non-tEPOR transgene (FIX gene for correction of hemophilia B, “Padua”) show a dramatic decrease in colony-forming ability, which is in line with many prior reports following CRISPR/AAV6-mediated genome editing in HSPCs (Dever, et al. Nature, 2016: Gomez-Ospina, et al. Nature Communications, 2019; Cromer, et al. Nature Medicine, 2021), we did not observe this effect in any of the tEPOR-integrating constructs. This indicates that tEPOR may initiate pro-survival signals that help to ameliorate the inherent toxicity of typical genome editing protocols.

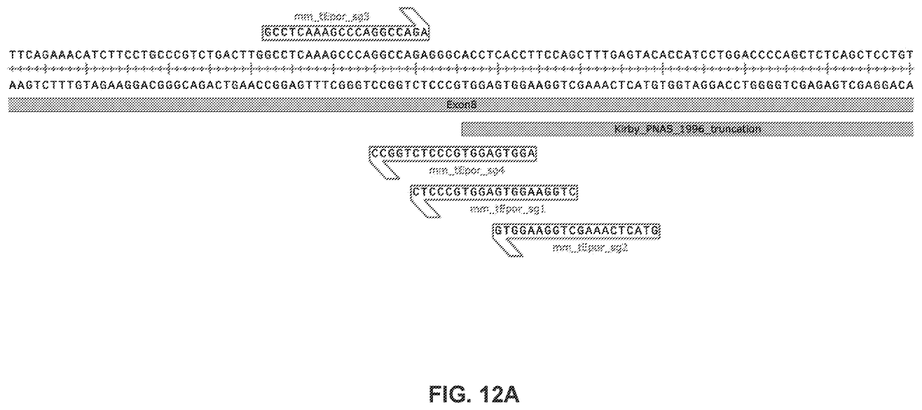

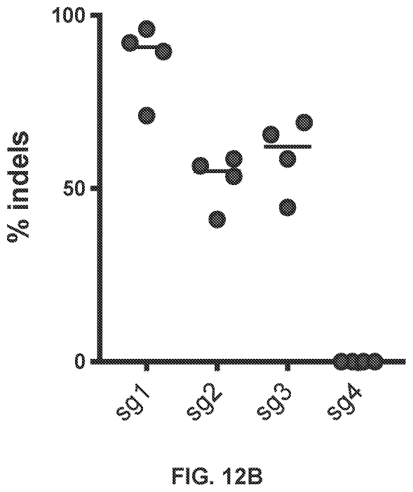

As the introduction of tEPOR into human HSPCs in vitro elicits consistent and robust enrichment, we next sought to determine the role of tEPOR in vivo following ex vivo editing and transplantation. Toward this end, we screened four gRNAs that introduced indels nearby the reported functional truncation of the endogenous mouse Epor gene (Kirby, et al. PNAS, 1996) (FIG. 12A). To do so, we delivered SpCas9 pre-complexed with each gRNA to mouse HSPCs and 4d later harvested gDNA and Sanger sequenced amplicons surrounding the predicted cleavage sites. Sanger traces were then used as input to TIDE (Brinkman, et al. Nucleic Acids Research, 2014) and we found sg1 initiated the highest frequency of indels at this locus (FIG. 12B).

### Example 2. Competitive HSC Transplantation of Edited Donor Mouse HSCs into Recipient Mice

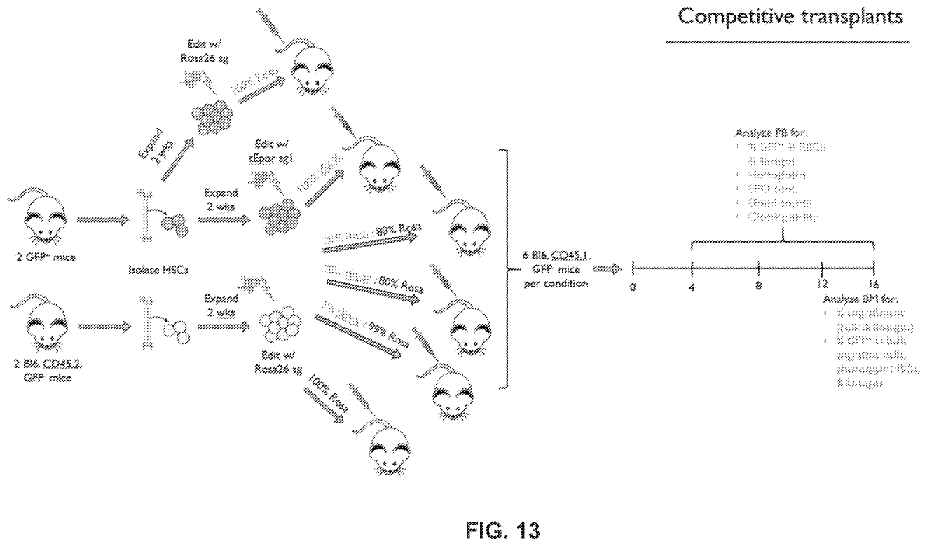

We have designed and initiated a large-scale competitive HSC transplantation experiment of edited donor mouse HSCs into recipient mice (FIG. 13). The workflow is comprised of HSC harvest from the bone marrow of GFP+ or GFP− B16 mice. A two-week HSC expansion is then used to generate a large number of HSCs for editing and subsequent transplantation via an established protocol (Wilkinson, et al. Nature, 2019). We then edit expanded HSCs with either the functional tEpor gRNA or the non-functional editing at the well-characterized Rosa26 locus. We then perform competitive transplants by mixing GFP+ and GFP− cells that either do or do not have Epor truncations and determine the following metrics over the course of 16 weeks post-transplantation: percentage of GFP+ cells in RBCs and other myeloid and lymphoid lineages, hemoglobin concentration, EPO concentration, blood counts, and clotting ability. At the conclusion of the study, we harvest bone marrow from these mice and determine: percent engraftment of GFP+ and GFP− populations in phenotypic HSCs and the various lineages. If a clear competitive advantage of tEpor-edited cells is not seen by week 8, we administer EPO injections to the mice in order to determine whether this yields any subsequent enrichment.

### Materials and Methods

### AAV6 Vector Design, Production, and Purification

All AAV6 vectors were cloned into the pAAV-MCS plasmid (Agilent Technologies, Santa Clara. CA, USA), which contains inverted terminal repeats (ITRs) derived from AAV2. Gibson Assembly Mastermix (New England Biolabs, Ipswich, MA, USA) was used for the creation of each vector as per manufacturer's instructions. Few modifications were made to the production of AAV6 vectors as described¹. 293T cells (Life Technologies, Carlsbad, CA, USA) were seeded in five 15 cm² dishes with 17×10⁶ cells per plate. 24 h later, each dish was transfected with a standard polyethylenimine (PEI) transfection of 6 μg ITR-containing plasmid and 22 μg pDGM6, which contains the AAV6 cap genes, AAV2 rep genes, and Ad5 helper genes. After a 48-72 h incubation, cells were purified using AAVPro Purification Kits (All Serotypes)(Takara Bio USA, Mountain View, CA, USA) as per manufacturer's instructions. AAV6 vectors were titered using ddPCR to measure number of vector genomes per μL as previously described².

### Culturing of CD34⁺ HSPCs

Human CD34⁺ HSPCs were cultured as previously described”. Healthy donor CD34⁺ HSPCs were sourced from fresh cord blood, frozen cord blood, and Plerixafor- and/or G-CSF-mobilized peripheral blood (AllCells, Alameda. CA, USA and STEMCELL Technologies, Vancouver, Canada). CD34⁺ HSPCs were cultured at 1×10⁵ cells/mL in StemSpan SFEM II (STEMCELL Technologies, Vancouver, Canada) base medium supplemented with stem cell factor (SCF)(100 ng/mL), thrombopoietin (TPO)(100 ng/mL), FLT3-ligand (100 ng/mL), IL-6 (100 ng/mL), UM171 (35 nM), streptomycin (20 mg/mL), and penicillin (20 U/mL). The cell incubator conditions were 37° C., 5% CO₂, and 5% O₂.

Genome editing of CD34⁺ HSPCs

Chemically modified Cas9 sgRNAs were purchased from Synthego (Menlo Park, CA, USA) and TriLink BioTechnologies (San Diego, CA, USA) and were purified by high-performance liquid chromatography (HPLC). The sgRNA modifications added were the 2′-O-methyl-3′-phosphorothioate at the three terminal nucleotides of the 5′ and 3′ ends described previously⁹. The target sequences for human sgRNAs were as follows: tEPORsg1: 5′-GCTCCCAGCTCTTGCGTCCA-3′; tEPORsg2: 5′-AGCTCAGGGCACAGTGTCCA-3′; HBAIsg: 5′-GGCAAGAAGCATGGCCACCG-3′; and CCR5sg: 5′-GCAGCATAGTGAGCCCAGAA-3′. The target sequences for mouse sgRNAs were as follows: tEpor_sg1: 5′-CTGGAAGGTGAGGTGCCCTC-3′: tEpor_sg2: 5′-GTACTCAAAGCTGGAAGGTG-3′; tEpor_sg3: 5′-GCCTCAAAGCCCAGGCCAGA-3′; and tEpor_sg4: 5′-AGGTGAGGTGCCCTCTGGCC-3′. All hi-fidelity variant¹⁰ Cas9 protein (SpyFi) was purchased from Aldevron, LLC (Fargo, ND, USA). The RNPs were complexed at a Cas9:sgRNA molar ratio of 1:2.5 at 25° C. for 10 min prior to electroporation. CD34” cells were resuspended in P3 buffer (Lonza, Basel, Switzerland) with complexed RNPs and electroporated using the Lonza 4D Nucleofector (program DZ-100). Cells were plated at 1×10⁵ cells/mL following electroporation in the cytokine-supplemented media described previously. Immediately following electroporation, AAV6 was supplied to the cells at 5×10⁵ vector genomes/cell based on titers determined by ddPCR.

Gene Targeting Analysis by/Flow Cytometry

For targeting analysis by flow cytometry, CD34⁺ HSPCs were harvested at d5 and erythrocytes at d16 post-targeting. Cells were analyzed for viability using Ghost Dye Red 780 (Tonbo Biosciences, San Diego, CA, USA) and reporter expression was assessed using the FACS Aria 11 system (BD Biosciences, San Jose, CA, USA). The data was subsequently analyzed using FlowJo (FlowJo LLC, Ashland, OR, USA).

In Vitro Differentiation of CD34⁺ HSPCs into Erythrocytes

Following targeting, HSPCs derived from healthy donors or polycythemia vera patients were cultured for 14-16d at 37° C. and 5% CO₂ in SFEM II medium (STEMCELL Technologies, Vancouver, Canada) as previously described^{11, 12}. SFEMII base medium was supplemented with 100 U/mL penicillin-streptomycin, 10 ng/mL SCF, 1 ng/mL IL-3 (PeproTech, Rocky Hill, NJ, USA), 3 U/mL erythropoietin (eBiosciences, San Diego, CA, USA), 200 μg/mL transferrin (Sigma-Aldrich, St. Louis, MO, USA), 3% antibody serum (heat-inactivated from Atlanta Biologicals, Flowery Branch, GA, USA), 2% human plasma (derived from umbilical cord blood), 10 μg/mL insulin (Sigma-Aldrich, St. Louis, MO, USA), and 3 U/mL heparin (Sigma-Aldrich, St. Louis, MO, USA). In the first phase, d0-7 (d0 being 2d post-targeting) of differentiation, cells were cultured at 1×10⁵ cells/mL. In the second phase, d7-10, cells were maintained at 1×10⁵ cells/mL, and IL-3 was removed from the culture. In the third phase, d11-16, cells were cultured at 1×10⁶ cells/mL, and transferrin was increased to 1 mg/mL within the culture medium.

### Immunophenotyping of Differentiated Erythrocytes

HSPCs subjected to the above erythrocyte differentiation were analyzed at d14 for erythrocyte lineage-specific markers using a FACS Aria II (BD Biosciences, San Jose, CA, USA). Edited and non-edited cells were analyzed by flow cytometry using the following antibodies: hCD45 V450 (H130: BD Biosciences, San Jose, CA, USA), CD34 APC (561; BioLegend, San Diego, CA, USA), CD71 PE-Cy7 (OKT9; Affymetrix, Santa Clara, CA, USA), and CD235a PE (GPA)(GA-R2: BD Biosciences, San Jose, CA, USA).

### Indel Frequency Analysis by TIDE

2-4d post-targeting, HSPCs were harvested and QuickExtract DNA extraction solution (Epicentre, Madison, WI, USA) was used to collect gDNA. Primers were then used to amplify the region surrounding the predicted cut site and/or deletion. PCR reactions were then run on a 1% agarose gel and appropriate bands were cut and gel-extracted using a GeneJET Gel Extraction Kit (Thermo Fisher Scientific, Waltham, MA, USA) according to manufacturer's instructions. Gel-extracted amplicons were then Sanger sequenced and resulting chromatograms were used as input for indel frequency analysis by TIDE as previously described”.

Allelic Targeting Analysis by ddPCR

2-4d post-targeting, HSPCs were harvested and QuickExtract DNA extraction solution (Epicentre, Madison, WI, USA) was used to collect gDNA, gDNA was then digested using BamHI-HF as per manufacturer's instructions (New England Biolabs, Ipswich, MA, USA). The percentage of targeted alleles within a cell population was measured by ddPCR using the following reaction mixture: 1-4 μL of digested gDNA input, 10 μL ddPCR SuperMix for Probes (No dUTP)(Bio-Rad, Hercules, CA, USA), primer/probes (1:3.6 ratio: Integrated DNA Technologies, Coralville, Iowa, USA), volume up to 20 μL with H₂O, ddPCR droplet were then generated following the manufacturer's instructions (Bio-Rad, Hercules, CA, USA): 20 μL of ddPCR reaction, 70 μL droplet generation oil, and 40 μL of droplet sample. Thermocycler (Bio-Rad, Hercules, CA, USA) settings were as follows: 1. 98° C. (10 min), 2. 94° C. (30 s), 3. 57.3° C. (30 s), 4. 72° C. (1.75 min)(return to step 2×40-50 cycles), 5. 98° C. (10 min). Analysis of droplet samples was done using the QX200 Droplet Digital PCR System (Bio-Rad, Hercules, CA, USA). To determine percentage of alleles targeted, the number of Poisson-corrected integrant copies/mL were divided by the number of Poisson-corrected reference DNA copies/mL.

mRNA Analysis

Following differentiation of HSPCs into erythrocytes, cells were harvested and RNA was extracted using RNeasy Plus Mini Kit (Qiagen, Hilden, Germany). Subsequently. cDNA was made from approximately 100 ng RNA using the iScript Reverse Transcription Supermix for RT-qPCR (Bio-Rad. Hercules, CA, USA). Expression levels of EPOR mRNA were quantified by ddPCR using specific primers and 6-FAM/ZEN/IBFQ-labelled hydrolysis probes purchased as custom-designed PrimeTime qPCR Assays from Integrated DNA Technologies (Coralvilla, IA, USA). To normalize for RNA input, levels of the RBC-specific reference gene GPA was determined in each sample using the following primers and HEX/ZEN/IBFQ-labelled hydrolysis probes purchased as custom-designed PrimeTime qPCR Assays from Integrated DNA Technologies (Coralvilla, IA, USA): forward: 5′-ATATGCAGCCACTCCTAGAGCTC-3′, reverse: 5′-CTGGTTCAGAGAAATGATGGGCA-3′, probe: 5′-AGGAAACCGGAGAAAGGGTA-3′, ddPCR reactions were created using the respective primers and probes and droplets were generated as described above. Thermocycler (Bio-Rad, Hercules, CA, USA) settings were as follows: 1. 98° C. (10 min), 2. 94° C. (30 s), 3. 59.4° C. (30 s), 4. 72° C. (30 s)(return to step 2-40-50 cycles), 5. 98° C. (10 min). Analysis of droplet samples was done using the QX200 Droplet Digital PCR System (Bio-Rad, Hercules, CA, USA). To determine relative expression levels, the number of Poisson-corrected HBA or HBB transgene copies/mL were divided by the number of GPA copies/mL.

### Methylcellulose CFU Assessment

2d post-targeting, HSPCs were counted and replicates of 250 and 500 cells were seeded into 6 well plates with MethoCult Optimum with and without EPO (STEMCELL Technologies, Vancouver, Canada). After 12-16d, colonies were scored based on external appearance using a STEMvision machine (STEMCELL Technologies, Vancouver, Canada) with automatic CFU scoring software. Colony counts were then manually reviewed for accuracy and correction in a blinded fashion.

### REFERENCES

1. Khan, I. F., Hirata, R. K. & Russell, D. W. AAV-mediated gene targeting methods for human cells. Nat Protoc 6, 482-501 (2011).
2. Aurnhammer, C. et al. Universal real-time PCR for the detection and quantification of adeno-associated virus serotype 2-derived inverted terminal repeat sequences. Hum Gene Ther Methods 23, 18-28 (2012).
3. Dever, D. P. et al. CRISPR/Cas9 beta-globin gene targeting in human haematopoietic stem cells. Nature 539, 384-389 (2016).
4. Charlesworth, C. T. et al. Priming Human Repopulating Hematopoietic Stem and Progenitor Cells for Cas9/sgRNA Gene Targeting. Mol Ther Nucleic Acids 12, 89-104 (2018).
5. Bak, R. O., Dever, D. P. & Porteus, M. H. CRISPR/Cas9 genome editing in human hematopoietic stem cells. Nat Protoc 13, 358-376 (2018).
6. Bak, R. O. et al. Multiplexed genetic engineering of human hematopoietic stem and progenitor cells using CRISPR/Cas9 and AAV6. Elife 6 (2017).
7. Cromer, M. K. et al. Global Transcriptional Response to CRISPR/Cas9-AAV6-Based Genome Editing in CD34(+) Hematopoietic Stem and Progenitor Cells. Mol Ther 26, 2431-2442 (2018).
8. Bak, R. O. & Porteus, M. H. CRISPR-Mediated Integration of Large Gene Cassettes Using AAV Donor Vectors. Cell Rep 20, 750-756 (2017).
9. Hendel. A. et al. Chemically modified guide RNAs enhance CRISPR-Cas genome editing in human primary cells. Nat Biotechnol 33, 985-989 (2015).
10. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
11. Dulmovits, B. M. et al. Pomalidomide reverses gamma-globin silencing through the transcriptional reprogramming of adult hematopoietic progenitors. Blood 127, 1481-1492 (2016).
12. Hu, J. et al. Isolation and functional characterization of human erythroblasts at distinct stages: implications for understanding of normal and disordered erythropoiesis in vivo. Blood 121, 3246-3253 (2013).
13. Brinkman, E. K., Chen, T., Amendola, M. & van Steensel, B. Easy quantitative assessment of genome editing by sequence trace decomposition. Nucleic Acids Res 42, e168 (2014).

### Example 3. Enrichment of Edited Cells Over the Course of Erythroid Differentiation by Combining a β-Thalassemia Correction Vector with a Truncated EPOR Transgene

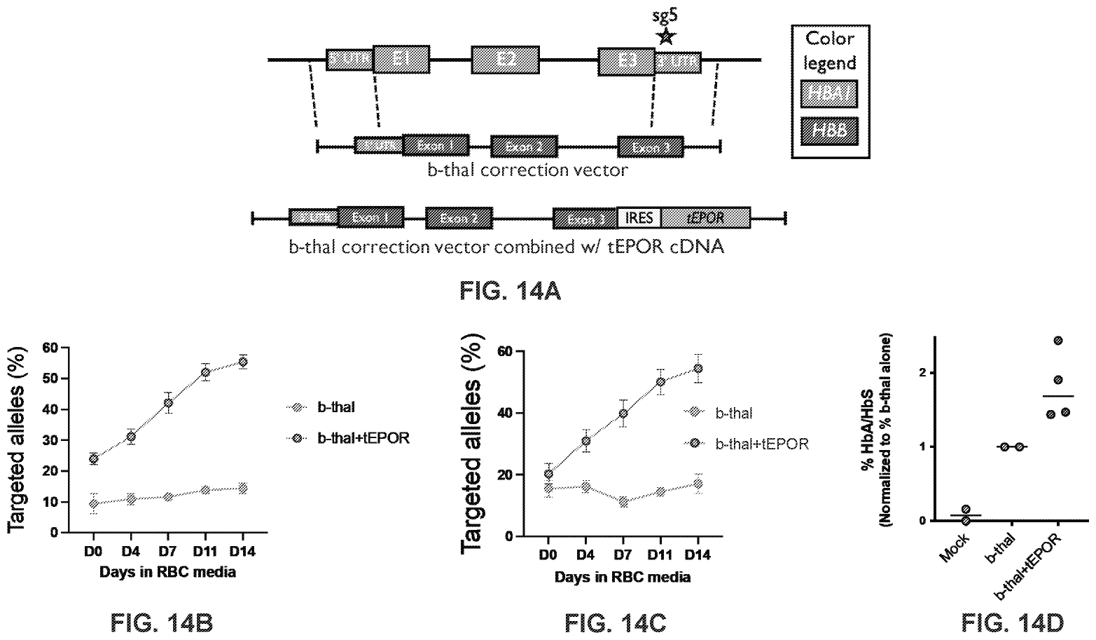

The present example describes use of the Cas9/AAV6 genome editing method to mediate site-specific integration of a β-thalassemia (β-thal) correction vector containing a full-length HBB transgene (from start codon to stop codon, including introns) combined with a cDNA encoding a truncated erythropoietin receptor (tEPOR) into the HBA1locus (FIG. 14A). As shown in Table 1, we found that the addition of the tEPOR cDNA caused a dramatic enrichment of edited alleles and edited red blood cells (RBCs) over the course of erythroid differentiation compared to the β-thal correction vector only for both wild-type (WT) primary human CD34⁺ HSPCs (FIG. 14B) and HSPCs from sickle cell disease (SCD) patients (FIG. 14C).

TABLE 1

| | edited alleles | | edited cells | |
|---|---|---|---|---|
| Vector | d0 | d14 | d0 | d14 |
| β-thal | 9% | 14% | 16% | 23% |
| β-thal + tEPOR | 24% | 55% | 34% | 88% |
| β-thal | 16% | 17% | 24% | 26% |
| β-thal + tEPOR | 20% | 55% | 30% | 85% |

We also found that the addition of the tEPOR cDNA to the correction vector caused a substantial increase in the formation of adult hemoglobin tetramers (HbA) compared to the correction vector only (FIG. 14D).

### Example 4. Enrichment of Edited Cells Over the Course of Erythroid Differentiation by Combining an α-Thalassemia Correction Vector with a Truncated EPOR Transgene

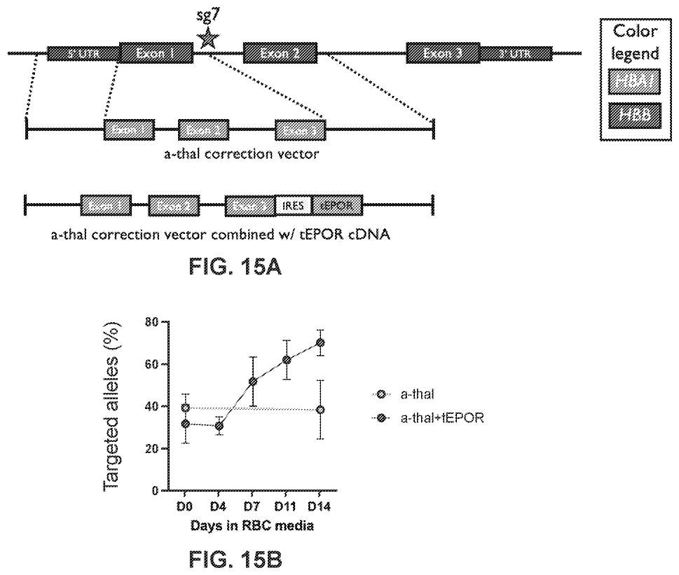

The present example describes use of the Cas9/AAV6 genome editing method to mediate site-specific integration of an α-thalassemia (a-thal) correction vector containing a full-length HBA1 transgene (from start codon to stop codon, including introns) combined with a cDNA encoding a truncated erythropoietin receptor (tEPOR) at the start codon of HBB gene (FIG. 15A). As shown in Table 2, we found that the addition of the tEPOR cDNA caused a dramatic enrichment of edited alleles and edited red blood cells (RBCs) over the course of erythroid differentiation compared to the correction vector only for wild-type (WT) primary human CD34⁺ HSPCs (FIG. 15B).

TABLE 2

| | edited alleles | | edited cells | |
|---|---|---|---|---|
| Vector | d0 | d14 | d0 | d14 |
| α-thal | 39% | 38% | 53% | 52% |
| α-thal + tEPOR | 32% | 70% | 42% | ~100% |

### 7. Exemplary Embodiments

Exemplary embodiments provided in accordance with the presently disclosed subject matter include, but are not limited to, the claims and the following embodiments:

1. A method of genetically modifying a hematopoietic stem and progenitor cell (HSPC), the method comprising:
introducing into the HSPC an RNA-guided nuclease and a guide RNA that specifically targets a sequence within the cytoplasmic domain-encoding region of the erythropoietin receptor (EPOR) locus in the genome of the cell; wherein
the RNA-guided nuclease cleaves the EPOR locus in the genome of the cell, resulting in the expression of a truncated erythropoietin receptor (tEPOR) in the cell and thereby generating a genetically modified HSPC; and wherein
the expression of the tEPOR increases the sensitivity of the cell to erythropoietin (EPO) and/or increases the proliferation of the cell in the presence of EPO as compared to the sensitivity and/or proliferation of a non-genetically modified HSPC.

2. The method of embodiment 1, wherein the cleavage of the EPOR locus by the RNA-guided nuclease creates an insertion or deletion (indel) that introduces a nonsense mutation into the EPOR locus.

3. The method of embodiment 1, further comprising introducing a donor template into the cell,
wherein the donor template comprises a first homologous region comprising complementarity to the EPOR locus upstream of the guide RNA target site, a second homologous region comprising complementarity to the EPOR locus downstream of the guide RNA target site, and a coding sequence located between the first and second homology regions that encodes a tEPOR, and wherein
the coding sequence is integrated into the cleaved EPOR locus, leading to the expression of the tEPOR in the cell.

4. The method of any one of embodiments 1 to 3, wherein the method further comprises isolating the HSPC from a subject prior to introducing the RNA-guided nuclease and the guide RNA into the cell.

5. The method of any one of embodiments 1 to 4, wherein expression of the tEPOR is driven by the endogenous EPOR promoter.

6. The method of any one of embodiments 1 to 5, wherein the tEPOR lacks a C-terminal portion of the EPOR cytoplasmic domain.

7. The method of any one of embodiments 1 to 6, wherein the first homology arm comprises the nucleotide sequence of SEQ ID NO:1 or a subsequence thereof, or a sequence comprising at least about 70%, 75%, 80%, 85%, 90%, 95%, 96%, 97%, 98%, 99%, or more identity to SEQ ID NO:1 or a subsequence thereof.

8. The method of any one of embodiments 1 to 7, wherein the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof.

9. The method of any one of embodiments 1 to 8, wherein the coding sequence encoding the truncated EPOR protein comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10.

10. The method of any one of embodiments 3 to 9, wherein the donor template comprises SEQ ID NO:3 or a subsequence thereof, or a sequence comprising at least about 70%, 75%, 80%, 85%, 90%, 95%, 96%, 97%, 98%, 99% or more identity to SEQ ID NO:3 or a subsequence thereof.

11. The method of any one of embodiments 1 to 10, wherein the target sequence of the guide RNA comprises the nucleotide sequence of SEQ ID NO: I1 or 12, or a nucleotide sequence comprising 1, 2, or 3 mismatches relative to SEQ ID NO: 11 or 12.

12. The method of any one of embodiments 1 to 11, wherein the guide RNA comprises one or more 2′-O-methyl-3′-phosphorothioate (MS) modifications.

13. The method of embodiment 12, wherein the one or more 2′-O-methyl-3′-phosphorothioate (MS) modifications are present at the three terminal nucleotides of the 5′ and 3′ ends of the guide RNA.

14. The method of any one of embodiments 1 to 13, wherein the RNA-guided nuclease is Cas9.

15. The method of embodiment 14, wherein the Cas9 is a High Fidelity Cas9.

16. The method of any one of embodiments 1 to 15, wherein the guide RNA and the RNA-guided nuclease are introduced into the HSPC as a ribonucleoprotein (RNP) complex by electroporation.

17. The method of any one of embodiments 3 to 16, wherein the donor template is introduced into the HSPC using a recombinant adeno-associated virus (rAAV) vector.

18. The method of embodiment 17, wherein the rAAV vector is a AAV6 vector.

19. The method of any one of embodiments 1 to 18, wherein the HSPC is genetically modified at a second locus other than EPOR, using an sgRNA targeting the second locus and a second homologous donor template comprising homology to the second locus.

20. The method of embodiment 19, wherein the second homologous donor template further comprises a therapeutic transgene.

21. The method of embodiment 20, wherein the therapeutic transgene is selected from the group consisting of HBA1, HBA2, HBB, PDGFB, IDUA, FIX, LDLR, and PAH.

22. The method of any one of embodiments 19 to 21, wherein the HSPC is isolated from a subject having a condition for which the genetic modification made at the second locus is beneficial.

23. The method of embodiment 22, wherein the condition is α-thalassemia. β-thalassemia, sickle cell disease, hemophilia B, phenylketonuria, Gaucher disease, or Krabbe disease.

24. The method of embodiment 22 or 23, wherein the genetically modified HSPC is reintroduced into the subject.

25. The method of embodiment 24, wherein the reintroduction of the genetically modified HSPC ameliorates one or more symptoms of the condition.

26. The method of embodiment 24 or 25, wherein the proportion of genetically modified HSPCs among red blood cells (RBCs) and/or one or more myeloid or lymphoid lineages in the subject increases over time.

27. The method of any one of embodiments 4 to 26, wherein the subject is a human.

28. A method of genetically modifying a hematopoietic stem and progenitor cell (HSPC), the method comprising:
introducing into the HSPC an RNA-guided nuclease, a donor template comprising a transgene encoding a truncated erythropoietin receptor (tEPOR), and a guide RNA that specifically targets a sequence within a safe harbor locus in the genome of the cell; wherein
the donor template comprises a first homologous region comprising complementarity to the safe harbor locus upstream of the guide RNA target site, a second homologous region comprising complementarity to the safe harbor locus downstream of the guide RNA target site, wherein the first and second homology regions flank the tEPOR transgene on the template, wherein
the RNA-guided nuclease cleaves the safe harbor locus in the genome of the cell and the transgene is integrated into the genome at the cleaved safe harbor locus, thereby generating a genetically modified HSPC; and wherein
the integrated transgene results in expression of the tEPOR in the genetically modified HSPC.

29. The method of embodiment 28, wherein expression of the tEPOR increases the sensitivity of the cell to erythropoietin (EPO) and/or increases the proliferation of the cell in the presence of EPO relative to the sensitivity and/or proliferation of a non-genetically modified HSPC.

30. The method of embodiment 28 or 29, wherein the method further comprises isolating the HSPC from a subject prior to introducing the RNA-guided nuclease, the donor template, and the guide RNA into the cell.

31. The method of any one of embodiments 28 to 30, wherein the transgene encoding the truncated EPOR comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10.

32. The method of any one of embodiments 28 to 31, wherein the guide RNA comprises one or more 2′-O-methyl-3′-phosphorothioate (MS) modifications.

33. The method of embodiment 32, wherein the one or more 2′-O-methyl-3′-phosphorothioate (MS) modifications are present at the three terminal nucleotides of the 5′ and 3′ ends of the guide RNA.

34. The method of any one of embodiments 28 to 33, wherein the RNA-guided nuclease is Cas9.

35. The method of embodiment 34, wherein the Cas9 is a High Fidelity Cas9.

36. The method of any one of embodiments 28 to 35, wherein the guide RNA and the RNA-guided nuclease are introduced into the HSPC as a ribonucleoprotein (RNP) complex by electroporation.

37. The method of any one of embodiments 28 to 36, wherein the donor template is introduced into the HSPC using a recombinant adeno-associated virus (rAAV) vector.

38. The method of embodiment 37, wherein the rAAV vector is a AAV6 vector.

39. The method of any one of embodiments 28 to 38, wherein the safe harbor locus is CCR5.

40. The method of embodiment 39, wherein the first homology arm comprises the nucleotide sequence of SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof.

41. The method of embodiment 39 or 40, wherein the second homology arm comprises the nucleotide sequence of SEQ ID NO:8 or a subsequence thereof, or a sequence comprising at least about 70%, 75%, 80%, 85%, 90%, 95%, 96%, 97%, 98%, 99%, or more identity to SEQ ID NO:8 or a subsequence thereof.

42. The method of any one of embodiments 39 to 41, wherein the target sequence of the guide RNA comprises the nucleotide sequence of SEQ ID NO: 14, or a nucleotide sequence comprising 1, 2, or 3 mismatches relative to SEQ ID NO: 14.

43. The method of any one of embodiments 39 to 42, wherein the donor template comprises SEQ ID NO:9 or a subsequence thereof, or a sequence comprising at least about 70%, 75%, 80%, 85%, 90%, 95%, 96%, 97%, 98%, 99% or more identity to SEQ ID NO:9 or a subsequence thereof.

44. The method of any one of embodiments 28 to 38, wherein the safe harbor locus is HBA1.

45. The method of embodiment 44, wherein the first homology arm comprises the nucleotide sequence of SEQ ID NO:4 or a subsequence thereof, or a sequence comprising at least about 70%, 75%, 80%, 85%, 90%, 95%, 96%, 97%, 98%, 99%, or more identity to SEQ ID NO:4 or a subsequence thereof.

46. The method of embodiment 44 or 45, wherein the second homology arm comprises the nucleotide sequence of SEQ ID NO:5 or a subsequence thereof, or a sequence comprising at least about 70%, 75%, 80%, 85%, 90%, 95%, 96%, 97%, 98%, 99%, or more identity to SEQ ID NO:5 or a subsequence thereof.

47. The method of any one of embodiments 44 to 46, wherein the target sequence of the guide RNA comprises the nucleotide sequence of SEQ ID NO: 13, or a nucleotide sequence comprising 1, 2, or 3 mismatches relative to SEQ ID NO: 13.

48. The method of any one of embodiments 44 to 47, wherein the donor template comprises SEQ ID NO:6 or a subsequence thereof, or a sequence comprising at least about 70%, 75%, 80%, 85%, 90%, 95%, 96%, 97%, 98%, 99% or more identity to SEQ ID NO:6 or a subsequence thereof.

49. The method of any one of embodiments 44 to 48, wherein the donor template further comprises a therapeutic transgene encoding a protein, and wherein the first and second homology regions flank the therapeutic transgene and the tEPOR transgene on the template.

50. The method of embodiment 49, wherein the donor template further comprises an internal ribosome entry site (IRES) or a sequence encoding a 2A cleavage peptide between the therapeutic transgene and the tEPOR transgene on the template.

51. The method of embodiment 49 or 50, wherein the HSPC comprises a mutation in an endogenous gene causative of a condition in a subject and the therapeutic transgene comprises a corrective sequence.

52. The method of any one of embodiments 49 to 51, wherein the therapeutic transgene is selected from the group consisting of HBB, PDGFB, IDUA, FIX, LDLR, and PAH.

53. The method of embodiment 51 or 52, wherein the therapeutic transgene is HBB and the condition is β-thalassemia or sickle cell disease.

54. The method of embodiment 51 or 52, wherein the therapeutic transgene is FIX and the condition is hemophilia B.

55. The method of embodiment 51 or 52, wherein the therapeutic transgene is PAH and the condition is phenylketonuria.

56. The method of any one of embodiments 49 to 55, wherein the HSPC comprises a population of HSPCs.

57. The method of embodiment 56, wherein expression of the therapeutic transgene and the tEPOR transgene causes an enrichment of genetically modified HSPCs in the population of HSPCs over the course of red blood cell differentiation as compared to expression of the therapeutic transgene in the absence of expression of the tEPOR transgene.

58. The method of any one of embodiments 49 to 57, wherein expression of the therapeutic transgene and the tEPOR transgene increases a level of adult hemoglobin tetramers in the genetically modified HSPC as compared to expression of the therapeutic transgene in the absence of expression of the tEPOR transgene.

59. The method of any one of embodiments 28 to 38, wherein the safe harbor locus is HBB.

60. The method of embodiment 59, wherein the first homology arm comprises the nucleotide sequence of SEQ ID NO:19 or a subsequence thereof, or a sequence comprising at least about 70%, 75%, 80%, 85%, 90%, 95%, 96%, 97%, 98%, 99%, or more identity to SEQ ID NO:19 or a subsequence thereof.

61. The method of embodiment 59 or 60, wherein the second homology arm comprises the nucleotide sequence of SEQ ID NO:20 or a subsequence thereof, or a sequence comprising at least about 70%, 75%, 80%, 85%, 90%, 95%, 96%, 97%, 98%, 99%, or more identity to SEQ ID NO:20 or a subsequence thereof.

62. The method of any one of embodiments 59 to 61, wherein the target sequence of the guide RNA comprises the nucleotide sequence of SEQ ID NO: 21, or a nucleotide sequence comprising 1, 2, or 3 mismatches relative to SEQ ID NO: 21.

63. The method of any one of embodiments 59 to 61, wherein the target sequence of the guide RNA comprises the nucleotide sequence of SEQ ID NO: 22 or 23, or a nucleotide sequence comprising 1, 2, or 3 mismatches relative to SEQ ID NO: 22 or 23.

64. The method of any one of embodiments 59 to 63, wherein the donor template further comprises a therapeutic transgene encoding a protein, and wherein the first and second homology regions flank the therapeutic transgene and the tEPOR transgene on the template.

65. The method of embodiment 64, wherein the donor template further comprises an internal ribosome entry site (IRES) or a sequence encoding a 2A cleavage peptide between the therapeutic transgene and the tEPOR transgene on the template.

66. The method of embodiment 64 or 65, wherein the HSPC comprises a mutation in an endogenous gene causative of a condition in a subject and the therapeutic transgene comprises a corrective sequence.

67. The method of any one of embodiments 64 to 66, wherein the therapeutic transgene is HBA1 or HBA2.

68. The method of embodiment 66 or 67, wherein the condition is a-thalassemia.

69. The method of any one of embodiments 64 to 68, wherein the HSPC comprises a population of HSPCs.

70. The method of embodiment 69, wherein expression of the therapeutic transgene and the tEPOR transgene causes an enrichment of genetically modified HSPCs in the population of HSPCs over the course of red blood cell differentiation as compared to expression of the therapeutic transgene in the absence of expression of the tEPOR transgene.

71. The method of any one of embodiments 64 to 70, wherein expression of the therapeutic transgene and the tEPOR transgene increases a level of adult hemoglobin tetramers in the genetically modified HSPC as compared to expression of the therapeutic transgene in the absence of expression of the tEPOR transgene.

72. The method of any one of embodiments 28 to 38, wherein the HSPC is genetically modified at a second locus other than the safe harbor locus using an sgRNA targeting the second locus and a second homologous donor template comprising homology to the second locus.

73. The method of embodiment 72, wherein the second homologous donor template further comprises a therapeutic transgene.

74. The method of embodiment 73, wherein the therapeutic transgene is selected from the group consisting of HBA1, HBA2, HBB, PDGFB, IDUA, FLY, LDLR, and PAH.

75. The method of any one of embodiments 72 to 74, wherein the HSPC is isolated from a subject having a condition for which the genetic modification introduced at the second locus is beneficial.

76. The method of embodiment 75, wherein the condition is α-thalassemia, β-thalassemia, sickle cell disease, hemophilia B, phenylketonuria, Gaucher disease, or Krabbe disease.

77. The method of any one of embodiments 51 to 58, 66 to 71, and 75 to 76, wherein the genetically modified HSPC is reintroduced into the subject.

78. The method of embodiment 77, wherein the reintroduction of the genetically modified HSPC ameliorates one or more symptoms of the condition.

79. The method of embodiment 77 or 78, wherein the proportion of genetically modified HSPCs among red blood cells (RBCs) and/or one or more myeloid or lymphoid lineages in the subject increases over time following the reintroduction of the HSPC into the subject.

80. The method of any one of embodiments 28 to 79, wherein the transgene comprises a heterologous promoter.

81. The method of embodiment 80, wherein the heterologous promoter is selected from the group consisting of EPOR, HBA1, PGK1, and UBC.

82. The method of any one of embodiments 30 to 81, wherein the subject is a human.

83. A genetically modified HSPC comprising a coding sequence encoding a tEPOR, wherein the genetically modified HSPC is generated using the method of any one of embodiments 1 to 82.

84. A donor template comprising a homology region comprising SEQ ID NO: 1 or SEQ ID NO: 2 or a subsequence thereof, or a nucleotide sequence comprising at least about 70%, 75%, 80%, 85%, 90%, 95%, 96%, 97%, 98%, 99% or more identity to SEQ ID NO: 1 or SEQ ID NO: 2 or a subsequence thereof.

85. A donor template comprising a nucleotide sequence selected from the group consisting of SEQ ID NOS: 3, 6, and 9 and subsequences thereof, or a nucleotide sequence comprising at least about 70%, 75%, 80%, 85%, 90%, 95%, 96%, 97%, 98%, 99% or more identity to any one of SEQ ID NOS: 3, 6, or 9, or a subsequence thereof.

86. A transgene comprising a nucleotide sequence encoding a tEPOR, wherein the nucleotide sequence comprises the sequence of SEQ ID NO:10, or a nucleotide sequence comprising at least about 70%, 75%, 80%, 85%, 90%, 95%, 96%, 97%, 98%, 99% or more identity to SEQ ID NO:10.

87. A guide RNA comprising a target sequence comprising SEQ ID NO: 11 or SEQ ID NO:12, or a sequence comprising 1, 2, or 3 mismatches with SEQ ID NO: 11 or SEQ ID NO:12.

88. An HSPC comprising the donor template of embodiment 84 or 85, the transgene of embodiment 86, and/or the guide RNA of embodiment 87.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### INFORMAL SEQUENCE LISTING

INFORMAL SEQUENCE LISTING

SEQ ID NO: 1
EPOR-integrating cassette left homology arm:
gagatgggggtctcactatgtttcctaggctggtcttgaactcctgggttcaaatgatcctcccacctcagcctcccaaagtactgggatt
atagtgctggtgtaaaccactgcacctggccatggccaggattaaagggagaatgaccaaggtatattgaactcctatgcacccttcaa
taccctgttccatttacccttttgtagggccttgctgatgcttcagccaaaacccctgtcccctggccctgatgtactcctctgcctccattgt
gatcacagggaccaagtgtatctgtgcctctatgactgggagtggagggggaattggtgagtattcaatgagtcatatctatgtaactatt
tatattggcttcaacagGGCTCTGAAGCAGAAGATCTGGCCTGGCATCCCGAGCCCAGAGA
GCGAGTTTGAAGGCCTCTTCACCACCCACAAGGGTAACTTCCAGgtaggtggcctggttgtc
ccctcagtgcctgggcttccctgcttcttgcagccaaactgcaggcctctctgagcaggttggtgctatttcttcagCTGTGGCTG
TACCAGAATGATGGCTGCCTGTGGTGGAGCCCCTGCACCCCCTTCACGGAGGACC
CACCTGCTTCCCTGGAAGTCCTCTCAGAGCGCTGCTGGGGGACGATGCAGGCAGT
GGAGCCGGGGACAGATGATGAGGGCCCCCTGCTGGAGCCAGTGGGCAGTGAGCA
TGCCCAGGATACCTATCTGGTGCTGGACAAATGGTTGCTGCCCCGGAACCCGCCC
AGTGAGGACCTCCCAGGGCCTGGTGGCAGTGTGGACATAGTGGCCATGGATGAA
GGCTCAGAAGCATCCTCCTGCTCATCTGCTTTGGCCTCGAAGCCCAGCCCAGAGG
GAGCCTCTGCTGCCAGCTTTGAGTACACTATCCTGGACCCCAGCTCCCAGCTCTT
GCGTCCATAGctcgagggcgcgccCGCTGATCAGCCTCGAC
SEQ ID NO: 2
EPOR-integrating cassette right homology arm:
ACACTGTGCCCTGAGCTGCCCCCTACCCCACCCCACCTAAAGTACCTGTACCTTG
TGGTATCTGACTCTGGCATCTCAACTGACTACAGCTCAGGGGACTCCCAGGGAGC
CCAAGGGGGCTTATCCGATGGCCCCTACTCCAACCCTTATGAGAACAGCCTTATC
CCAGCCGCTGAGCCTCTGCCCCCCAGCTATGTGGCTTGCTCTTAGGACACCAGGC
TGCAGATGATCAGGGATCCAATATGACTCAGAGAACCAGTGCAGACTCAAGACT
TATGGAACAGGGATGGCGAGGCCTCTCTCAGGAGCAGGGGCATTGCTGATTTTGT
CTGCCCAATCCATCCTGCTCAGGAAACCACAACCTTGCAGTATTTTTAAATATGT
ATAGTTTTTTTTTGTATCTATATATATATATACACATATGTATGTAAGTTTTTCTAC
CATGATTTCTACAAACACCCTTTAAGTCCCATCTTCCCCTGGGCATAGGCCATAG
GGATAGAAGTTAAAGTTCTTGAGCTTATTCAGAAGCTGGATCTGCAATCTGAATG
CTACTCATAACATAACAAAATAGTATGTTAAACAGCTCTTAAATCTTACTGGCTT
ACCACATTAAATGATTTCTCTCTCCTAACTCAGCTCAAATGGGCAGCCATCCATG
GGATGAGTCAGAGGTTCAGACTCTTCCAGTCTGTAGCTCTACCTTCTCTTAGGGT
ACTTAGATGGATCCCCTGTTCTACAAACTGCCAGTCAGCAAGGGAAGAAAAAGG
GCAGCAATGACCCTCAATGGGCCATTTGAGGGATCTGGCCTGGAAATGGGCTTCC
TCTCTTCTTCTCACACCTCACTGGCTGGAAACAGTCACATGACCCCAGTCACATG
AAAGGCCAGGAAACTTAGTTTAGCTGTACACCCAGGAAGGGCAAAGCTGTTTAA
GGGCCACTAGCTAGTCTCTGCCACTAAT
SEQ ID NO: 3
bGH-UbC-GFP cassette that introduces truncation of endogenous locus:
CTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCAT
CGCATTGTCTGAGTAGGTGTCATTCTATTCTGGGGGGTGGGGGGGGCAGGACAG
CAAGGGGGAGGATTGGGAAGACAATAGCAGGCATGCTGGGGATGCGGTGGGCT
CTATGGCTTCTGAGGCGGAAAGAACGTTTCGCGCCattaccctgttatccctaGGCCTCCGCG
CCGGGTTTTGGCGCCTCCCGCGGGCGCCCCCCTCCTCACGGCGAGCGCTGCCACG
TCAGACGAAGGGCGCAGCGAGCGTCCTGATCCTTCCGCCCGGACGCTCAGGACA
GCGGCCCGCTGCTCATAAGACTCGGCCTTAGAACCCCAGTATCAGCAGAAGGAC
ATTTTAGGACGGGACTTGGGTGACTCTAGGGCACTGGTTTTCTTTCCAGAGAGCG
GAACAGGCGAGGAAAAGTAGTCCCTTCTCGGCGATTCTGCGGAGGGATCTCCGT
GGGGCGGTGAACGCCGATGATTATATAAGGACGCGCCGGGTGTGGCACAGCTAG
TTCCGTCGCAGCCGGGATTTGGGTCGCGGTTCTTGTTTGTGGATCGCTGTGATCGT
CACTTGGTGAGTAGCGGGCTGCTGGGCTGGCCGGGGCTTTCGTGGCCGCCGGGCC
GCTCGGTGGGACGGAAGCGTGTGGAGAGACCGCCAAGGGCTGTAGTCTGGGTCC
GCGAGCAAGGTTGCCCTGAACTGGGGGTTGGGGGGAGCGCAGCAAAATGGCGGC
TGTTCCCGAGTCTTGAATGGAAGACGCTTGTGAGGCGGGCTGTGAGGTCGTTGAA
ACAAGGTGGGGGGCATGGTGGGCGGCAAGAACCCAAGGTCTTGAGGCCTTCGCT
AATGCGGGAAAGCTCTTATTCGGGTGAGATGGGCTGGGGCACCATCTGGGGACC
CTGACGTGAAGTTTGTCACTGACTGGAGAACTCGGTTTGTCGTCTGTTGCGGGGG
CGGCAGTTATGGCGGTGCCGTTGGGCAGTGCACCCGTACCTTTGGGAGCGCGCGC
CCTCGTCGTGTCGTGACGTCACCCGTTCTGTTGGCTTATAATGCAGGGTGGGGCC
ACCTGCCGGTAGGTGTGCGGTAGGCTTTTCTCCGTCGCAGGACGCAGGGTTCGGG
CCTAGGGTAGGCTCTCCTGAATCGACAGGCGCCGGACCTCTGGTGAGGGGAGGG
ATAAGTGAGGCGTCAGTTTCTTTGGTCGGTTTTATGTACCTATCTTCTTAAGTAGC
TGAAGCTCCGGTTTTGAACTATGCGCTCGGGGTTGGCGAGTGTGTTTTGTGAAGT
TTTTTAGGCACCTTTTGAAATGTAATCATTTGGGTCAATATGTAATTTTCAGTGTT
AGACTAGTAAATTGTCCGCTAAATTCTGGCCGTTTTTGGCTTTTTTGTTAGACggtac
cgagctcttcgaaggatccatcgccaccATGCCCGCCATGAAGATCGAGTGCCGCATCACCGGCA
CCCTGAACGGCGTGGAGTTCGAGCTGGTGGGCGGCGGAGAGGGCACCCCCGAGC
AGGGCCGCATGACCAACAAGATGAAGAGCACCAAAGGCGCCCTGACCTTCAGCC
CCTACCTGCTGAGCCACGTGATGGGCTACGGCTTCTACCACTTCGGCACCTACCC
CAGCGGCTACGAGAACCCCTTCCTGCACGCCATCAACAACGGCGGCTACACCAA
CACCCGCATCGAGAAGTACGAGGACGGCGGCGTGCTGCACGTGAGCTTCAGCTA
CCGCTACGAGGCCGGCCGCGTGATCGGCGACTTCAAGGTGGTGGGCACCGGCTT
CCCCGAGGACAGCGTGATCTTCACCGACAAGATCATCCGCAGCAACGCCACCGT
GGAGCACCTGCACCCCATGGGCGATAACGTGCTGGTGGGCAGCTTCGCCCGCAC
CTTCAGCCTGCGCGACGGCGGCTACTACAGCTTCGTGGTGGACAGCCACATGCAC
TTCAAGAGCGCCATCCACCCCAGCATCCTGCAGAACGGGGGCCCCATGTTCGCCT
TCCGCCGCGTGGAGGAGCTGCACAGCAACACCGAGCTGGGCATCGTGGAGTACC
AGCACGCCTTCAAGACCCCCATCGCCTTCGCCAGATCTCGAGTCTAG
SEQ ID NO: 4
HBA1-integrating cassette left homology arm:
gctccagccggttccagctattgctttgtttacctgtttaaccagtatttacctagcaagtcttccatcagatagcatttggagagctggggg
tgtcacagtgaaccacgacctctaggccagtgggagagtcagtcacacaaactgtgagtccatgacttggggcttagccagcaccca
ccaccccacgcgccaccccacaaccccgggtagaggagtctgaatctggagccgcccccagcccagccccgtgctttttgcgtcct
ggtgtttattccttcccggtgcctgtcactcaagcacactagtgactatcgccagagggaaagggagctgcaggaagcgaggctgga
gagcaggaggggctctgcgcagaaattcttttgagttcctatgggccagggcgtccgggtgcgcgcattcctctccgccccaggattg
ggcgaagcctcccggctcgcactcgctcgcccgtgtgttccccgatcccgctggagtcgatgcgcgtccagcgcgtgccaggccgg
ggcgggggtgcgggctgactttctccctcgctagggacgctccggcgcccgaaaggaaagggggcgctgcgctccggggtgcac
gagccgacagcgcccgaccccaacgggccggccccgccagcgccgctaccgccctgcccccgggcgagcgggatgggggg
agtggagtggcgggtggagggtggagacgtcctggcccccgccccgcgtgcacccccaggggaggccgagcccgccgcccgg
ccccgcgcaggccccgcccgggactcccctgcggtccaggccgcgccccgggctccgcgccagccaatgagcgccgcccggcc
gggcgtgcccccgcgccccaagcataaaccctggcgcgctcgcggcccggcactcttctggtccccacagactcagagagaaccc
acc
SEQ ID NO: 5
HBA1-integrating cassette right homology arm:
tggccatgcttcttgccccttgggcctccccccagcccctcctccccttcctgcacccgtacccccgtggtctttgaataaagtctgagtg
ggcggcagcctgtgtgtgcctgagttttttccctcagcaaacgtgccaggcatgggcgtggacagcagctgggacacacatggctag
aacctctctgcagctggatagggtaggaaaaggcaggggcgggaggaggggatggaggagggaaagtggagccaccgcgaagt
ccagctggaaaaacgctggaccctagagtgctttgaggatgcatttgctctttcccgagttttattcccagacttttcagattcaatgcagg
tttgctgaaataatgaatttatccatctttacgtttctgggcactctgtgccaagaactggctggctttctgcctgggacgtcactggtttccc
agaggtcctcccacatatggggggggtaggtcagagaagtcccactccagcatggctgcattgatcccccatcgttcccactagtct
ccgtaaaacctcccagatacaggcacagtctagatgaaatcagggggggggtgcaactgcaggccccaggcaattcaatagggg
ctctactttcacccccaggtcaccccagaatgctcacacaccagacactgacgccctggggctgtcaagatcaggcgtttgtctctggg
cccagctcagggcccagctcagcacccactcagctcccctgaggctggggagcctgtcccattgcgactggagaggagagcgggg
ccacagaggcctggctagaaggtcccttctccctggtgtgtgttttctctctgctgagcaggcttgcagtgcctggggtatca
SEQ ID NO: 6
TEPOR cDNA-T2A-YFP cassette for integration at HBA1:
ATGGACCACCTCGGGGCGTCCCTCTGGCCCCAGGTCGGCTCCCTTTGTCTCCTGCT
CGCTGGGGCCGCCTGGGCGCCCCCGCCTAACCTCCCGGACCCCAAGTTCGAGAG
CAAAGCGGCCTTGCTGGCGGCCCGGGGGCCCGAAGAGCTTCTGTGCTTCACCGA
GCGGTTGGAGGACTTGGTGTGTTTCTGGGAGGAAGCGGCGAGCGCTGGGGTGGG
CCCGGGCAACTACAGCTTCTCCTACCAGCTCGAGGATGAGCCATGGAAGCTGTGT
CGCCTGCACCAGGCTCCCACGGCTCGTGGTGCGGTGCGCTTCTGGTGTTCGCTGC
CTACAGCCGACACGTCGAGCTTCGTGCCCCTAGAGTTGCGCGTCACAGCAGCCTC
CGGCGCTCCGCGATATCACCGTGTCATCCACATCAATGAAGTAGTGCTCCTAGAC
GCCCCCGTGGGGCTGGTGGCGCGGTTGGCTGACGAGAGCGGCCACGTAGTGTTG
CGCTGGCTCCCGCCGCCTGAGACACCCATGACGTCTCACATCCGCTACGAGGTGG
ACGTCTCGGCCGGCAACGGCGCAGGGAGCGTACAGAGGGTGGAGATCCTGGAGG
GCCGCACCGAGTGTGTGCTGAGCAACCTGCGGGGCCGGACGCGCTACACCTTCG
CCGTCCGCGCGCGTATGGCTGAGCCGAGCTTCGGCGGCTTCTGGAGCGCCTGGTC
GGAGCCTGTGTCGCTGCTGACGCCTAGCGACCTGGACCCCCTCATCCTGACGCTC
TCCCTCATCCTCGTGGTCATCCTGGTGCTGCTGACCGTGCTCGCGCTGCTCTCCCA
CCGCCGGGCTCTGAAGCAGAAGATCTGGCCTGGCATCCCGAGCCCAGAGAGCGA
GTTTGAAGGCCTCTTCACCACCCACAAGGGTAACTTCCAGCTGTGGCTGTACCAG
AATGATGGCTGCCTGTGGTGGAGCCCCTGCACCCCCTTCACGGAGGACCCACCTG
CTTCCCTGGAAGTCCTCTCAGAGCGCTGCTGGGGGACGATGCAGGCAGTGGAGC
CGGGGACAGATGATGAGGGCCCCCTGCTGGAGCCAGTGGGCAGTGAGCATGCCC
AGGATACCTATCTGGTGCTGGACAAATGGTTGCTGCCCCGGAACCCGCCCAGTGA
GGACCTCCCAGGGCCTGGTGGCAGTGTGGACATAGTGGCCATGGATGAAGGCTC
AGAAGCATCCTCCTGCTCATCTGCTTTGGCCTCGAAGCCCAGCCCAGAGGGAGCC
TCTGCTGCCAGCTTTGAGTACACTATCCTGGACCCCAGCTCCCAGCTCTTGCGTCC
Agagggcaggggcagcctgctgacctgcggcgacgtggaggagaaccccggccccatgggtgagcaagggcgaggagctgttca
ccggggtggtgcccatcctggtcgagctggacggcgacgtaaacggccacaagttcagcgtgtccggcgagggcgagggcgatg
ccacctaggcaagctgaccctgaagttcatctgcaccaccggcaagctgcccgtgccctggcccaccctcgtgaccaccttcggct
acggcctgatgtgcttcgcccgctaccccgaccacatgaagcagcacgacttcttcaagtccgccatgcccgaaggctacgtccagg
agcgcaccatcttcttcaaggacgacggcaactacaagacccgcgccgaggtgaagttcgagggcgacaccctggtgaaccgcatc
gagctgaagggcatcgacttcaaggaggacggcaacatcctggggcacaagctggagtacaactacaacagccacaacgtctatat
catggccgacaagcagaagaacggcatcaaggtgaacttcaagatccgccacaacatcgaggacggcagcgtgcagctcgccgac
cactaccagcagaacacccccatcggcgacggccccgtgctgctgcccgacaaccactacctgagctaccagtccaagctgagcaa
agaccccaacgagaagcgcgatcacatggtcctgctggagttcgtgaccgccgccgggatcactctcggcatggacgagctgtaca
agtaa
SEQ ID NO: 7
CCR5-integrating cassette left homology arm:
AAGAGAGTTAATTCAATGTAGACATCTATGTAGGCAATTAAAAACCTATTGATGT
ATAAAACAGTTTGCATTCATGGAGGGCAACTAAATACATTCTAGGACTTTATAAA
AGATCACTTTTTATTTATGCACAGGGTGGAACAAGATGGATTATCAAGTGTCAAG
TCCAATCTATGACATCAATTATTATACATCGGAGCCCTGCCAAAAAATCAATGTG
AAGCAAATCGCAGCCCGCCTCCTGCCTCCGCTCTACTCACTGGTGTTCATCTTTGG
TTTTGTGGGCAACATGCTGGTCATCCTCATCCTGATAAACTGCAAAAGGCTGAAG
AGCATGACTGACATCTACCTGCTCAACCTGGCCATCTCTGACCTGTTTTTCCTTCT
TACTGTCCCCTTC
SEQ ID NO: 8
CCR5-integrating cassette right homology arm:
tgggctcactatgctgccgcccagtgggactttggaaatacaatgtgtcaactcttgacagggctctattttataggcttcttctctggaatc
ttcttcatcatcctcctgacaatcgataggtacctggctgtcgtccatgctgtgtttgctttaaaagccaggacggtcacctttggggtggt
gacaagtgtgatcacttgggtggtggctgtgtttgcgtctctcccaggaatcatctttaccagatctcaaaaagaaggtcttcattacacct
gcagctctcattttccatacagtcagtatcaattctggaagaatttccagacattaaagatagtcatcttggggctggtcctgccgctgcttg
tcatggtcatctgctactcgggaatcctaa
SEQ ID NO: 9
UbC-tEPOR cDNA-T2A-YFP cassette for integration at CCR5:
attaccctgttatccctaGGCCTCCGCGCCGGGTTTTGGCGCCTCCCGCGGGCGCCCCCCTCC
TCACGGCGAGCGCTGCCACGTCAGACGAAGGGCGCAGCGAGCGTCCTGATCCTT
CCGCCCGGACGCTCAGGACAGCGGCCCGCTGCTCATAAGACTCGGCCTTAGAAC
CCCAGTATCAGCAGAAGGACATTTTAGGACGGGACTTGGGTGACTCTAGGGCAC
TGGTTTTCTTTCCAGAGAGCGGAACAGGCGAGGAAAAGTAGTCCCTTCTCGGCGA
TTCTGCGGAGGGATCTCCGTGGGGCGGTGAACGCCGATGATTATATAAGGACGC
GCCGGGTGTGGCACAGCTAGTTCCGTCGCAGCCGGGATTTGGGTCGCGGTTCTTG
TTTGTGGATCGCTGTGATCGTCACTTGGTGAGTAGCGGGCTGCTGGGCTGGCCGG
GGCTTTCGTGGCCGCCGGGCCGCTCGGTGGGACGGAAGCGTGTGGAGAGACCGC
CAAGGGCTGTAGTCTGGGTCCGCGAGCAAGGTTGCCCTGAACTGGGGGTTGGGG
GGAGCGCAGCAAAATGGCGGCTGTTCCCGAGTCTTGAATGGAAGACGCTTGTGA
GGCGGGCTGTGAGGTCGTTGAAACAAGGTGGGGGGCATGGTGGGCGGCAAGAA
CCCAAGGTCTTGAGGCCTTCGCTAATGCGGGAAAGCTCTTATTCGGGTGAGATGG
GCTGGGGCACCATCTGGGGACCCTGACGTGAAGTTTGTCACTGACTGGAGAACTC
GGTTIGTCGTCTGTTGCGGGGGCGGCAGTTATGGCGGTGCCGTTGGGCAGTGCAC
CCGTACCTTTGGGAGCGCGCGCCCTCGTCGTGTCGTGACGTCACCCGTTCTGTTG
GCTTATAATGCAGGGTGGGGCCACCTGCCGGTAGGTGTGCGGTAGGCTTTTCTCC
GTCGCAGGACGCAGGGTTCGGGCCTAGGGTAGGCTCTCCTGAATCGACAGGCGC
CGGACCTCTGGTGAGGGGAGGGATAAGTGAGGCGTCAGTTTCTTTGGTCGGTTTT
ATGTACCTATCTTCTTAAGTAGCTGAAGCTCCGGTTTTGAACTATGCGCTCGGGG
TTGGCGAGTGTGTTTTGTGAAGTTTTTTAGGCACCTTTTGAAATGTAATCATTTGG
GTCAATATGTAATTTTCAGTGTTAGACTAGTAAATTGTCCGCTAAATTCTGGCCGT
TTTTGGCTTTTTTGTTAGACggtaccgagctcttcgaaggatccatcgccaccATGGACCACCTCGGG
GCGTCCCTCTGGCCCCAGGTCGGCTCCCTTTGTCTCCTGCTCGCTGGGGCCGCCTG
GGCGCCCCCGCCTAACCTCCCGGACCCCAAGTTCGAGAGCAAAGCGGCCTTGCT
GGCGGCCCGGGGGCCCGAAGAGCTTCTGTGCTTCACCGAGCGGTTGGAGGACTT
GGTGTGTTTCTGGGAGGAAGCGGCGAGCGCTGGGGTGGGCCCGGGCAACTACAG
CTTCTCCTACCAGCTCGAGGATGAGCCATGGAAGCTGTGTCGCCTGCACCAGGCT
CCCACGGCTCGTGGTGCGGTGCGCTTCTGGTGTTCGCTGCCTACAGCCGACACGT
CGAGCTTCGTGCCCCTAGAGTTGCGCGTCACAGCAGCCTCCGGCGCTCCGCGATA
TCACCGTGTCATCCACATCAATGAAGTAGTGCTCCTAGACGCCCCCGTGGGGCTG
GTGGCGCGGTTGGCTGACGAGAGCGGCCACGTAGTGTTGCGCTGGCTCCCGCCG
CCTGAGACACCCATGACGTCTCACATCCGCTACGAGGTGGACGTCTCGGCCGGCA
ACGGCGCAGGGAGCGTACAGAGGGTGGAGATCCTGGAGGGCCGCACCGAGTGT
GTGCTGAGCAACCTGCGGGGCCGGACGCGCTACACCTTCGCCGTCCGCGCGCGT
ATGGCTGAGCCGAGCTTCGGCGGCTTCTGGAGCGCCTGGTCGGAGCCTGTGTCGC
TGCTGACGCCTAGCGACCTGGACCCCCTCATCCTGACGCTCTCCCTCATCCTCGTG
GTCATCCTGGTGCTGCTGACCGTGCTCGCGCTGCTCTCCCACCGCCGGGCTCTGA
AGCAGAAGATCTGGCCTGGCATCCCGAGCCCAGAGAGCGAGTTTGAAGGCCTCT
TCACCACCCACAAGGGTAACTTCCAGCTGTGGCTGTACCAGAATGATGGCTGCCT
GTGGTGGAGCCCCTGCACCCCCTTCACGGAGGACCCACCTGCTTCCCTGGAAGTC
CTCTCAGAGCGCTGCTGGGGGACGATGCAGGCAGTGGAGCCGGGGACAGATGAT
GAGGGCCCCCTGCTGGAGCCAGTGGGCAGTGAGCATGCCCAGGATACCTATCTG
GTGCTGGACAAATGGTTGCTGCCCCGGAACCCGCCCAGTGAGGACCTCCCAGGG
CCTGGTGGCAGTGTGGACATAGTGGCCATGGATGAAGGCTCAGAAGCATCCTCC
TGCTCATCTGCTTTGGCCTCGAAGCCCAGCCCAGAGGGAGCCTCTGCTGCCAGCT
TTGAGTACACTATCCTGGACCCCAGCTCCCAGCTCTTGCGTCCAgagggcaggggcagcct
gctgacctgcggcgacgtggaggagaaccccggccccatggtgagcaagggcgaggagctgttcaccggggggtgcccatcct
ggtcgagctggacggcgacgtaaacggccacaagttcagcgtgtccggcgagggcgagggcgatgccacctacggcaagctgac
cctgaagttcatctgcaccaccggcaagctgcccgtgccctggcccaccctcgtgaccaccttcggctacggcctgatgtgcttcgcc
cgctaccccgaccacatgaagcagcacgacttcttcaagtccgccatgcccgaaggctacgtccaggagcgcaccatcttcttcaag
gacgacggcaactacaagacccgcgccgaggtgaagttcgagggcgacaccctggtgaaccgcatcgagctgaagggcatcgac
ttcaaggaggacggcaacatcctggggcacaagctggagtacaactacaacagccacaacgtctatatcatggccgacaagcagaa
gaacggcatcaaggtgaacttcaagatccgccacaacatcgaggacggcagcgtgcagctcgccgaccactaccagcagaacacc
cccatcggcgacggccccgtgctgctgcccgacaaccactacctgagctaccagtccaagctgagcaaagaccccaacgagaagc
gcgatcacatggtcctgctggagttcgtgaccgccgccgggatcactctcggcatggacgagctgtacaagtaactgtgccttctagtt
gccagccatctgttgtttgcccctcccccgtgccttccttgaccctggaaggtgccactcccactgtcctttcctaataaaatgaggaaatt
gcatcgcattgtctgagtaggtgtcattctattctggggggtggggggggcaggacagcaagggggaggattgggaagacaatagc
aggcatgctggggatgcggtgggctctatgg
SEQ ID NO: 10
tEPOR cDNA:
ATGGACCACCTCGGGGCGTCCCTCTGGCCCCAGGTCGGCTCCCTTTGTCTCCTGCT
CGCTGGGGCCGCCTGGGCGCCCCCGCCTAACCTCCCGGACCCCAAGTTCGAGAG
CAAAGCGGCCTTGCTGGCGGCCCGGGGGCCCGAAGAGCTTCTGTGCTTCACCGA
GCGGTTGGAGGACTTGGTGTGTTTCTGGGAGGAAGCGGCGAGCGCTGGGGTGGG
CCCGGGCAACTACAGCTTCTCCTACCAGCTCGAGGATGAGCCATGGAAGCTGTGT
CGCCTGCACCAGGCTCCCACGGCTCGTGGTGCGGTGCGCTTCTGGTGTTCGCTGC
CTACAGCCGACACGTCGAGCTTCGTGCCCCTAGAGTTGCGCGTCACAGCAGCCTC
CGGCGCTCCGCGATATCACCGTGTCATCCACATCAATGAAGTAGTGCTCCTAGAC
GCCCCCGTGGGGCTGGTGGCGCGGTTGGCTGACGAGAGCGGCCACGTAGTGTTG
CGCTGGCTCCCGCCGCCTGAGACACCCATGACGTCTCACATCCGCTACGAGGTGG
ACGTCTCGGCCGGCAACGGCGCAGGGAGCGTACAGAGGGTGGAGATCCTGGAGG
GCCGCACCGAGTGTGTGCTGAGCAACCTGCGGGGCCGGACGCGCTACACCTTCG
CCGTCCGCGCGCGTATGGCTGAGCCGAGCTTCGGCGGCTTCTGGAGCGCCTGGTC
GGAGCCTGTGTCGCTGCTGACGCCTAGCGACCTGGACCCCCTCATCCTGACGCTC
TCCCTCATCCTCGTGGTCATCCTGGTGCTGCTGACCGTGCTCGCGCTGCTCTCCCA
CCGCCGGGCTCTGAAGCAGAAGATCTGGCCTGGCATCCCGAGCCCAGAGAGCGA
GTTTGAAGGCCTCTTCACCACCCACAAGGGTAACTTCCAGCTGTGGCTGTACCAG
AATGATGGCTGCCTGTGGTGGAGCCCCTGCACCCCCTTCACGGAGGACCCACCTG
CTTCCCTGGAAGTCCTCTCAGAGCGCTGCTGGGGGACGATGCAGGCAGTGGAGC
CGGGGACAGATGATGAGGGCCCCCTGCTGGAGCCAGTGGGCAGTGAGCATGCCC
AGGATACCTATCTGGTGCTGGACAAATGGTTGCTGCCCCGGAACCCGCCCAGTGA
GGACCTCCCAGGGCCTGGTGGCAGTGTGGACATAGTGGCCATGGATGAAGGCTC
AGAAGCATCCTCCTGCTCATCTGCTTTGGCCTCGAAGCCCAGCCCAGAGGGAGCC
TCTGCTGCCAGCTTTGAGTACACTATCCTGGACCCCAGCTCCCAGCTCTTGCGTCC
A
SEQ ID NO: 11
tEpor_sg1 target sequence
5′-GCTCCCAGCTCTTGCGTCCA-3′
SEQ ID NO: 12
tEpor_sg2 target sequence
5′-AGCTCAGGGCACAGTGTCCA-3′
SEQ ID NO: 13
HBA1 sg5 target sequence
5′-GGCAAGAAGCATGGCCACCG-3′
SEQ ID NO: 14
CCR5 sg target sequence
5′-GCAGCATAGTGAGCCCAGAA~3′
SEQ ID NO: 15
mm_tEpor_sg1
5′-CTGGAAGGTGAGGTGCCCTC-3′
SEQ ID NO: 16
mm_tEpor_sg2
5′-GTACTCAAAGCTGGAAGGTG-3′
SEQ ID NO: 17
mm_tEpor_sg3
5′-GCCTCAAAGCCCAGGCCAGA-3′
SEQ ID NO: 18
mm_tEpor_sg4
5′-AGGTGAGGTGCCCTCTGGCC-3′
SEQ ID NO: 19
HBB left homology arm
ATTAGTCCAGGCAGAAACAGTTAGATGTCCCCAGTTAACCTCCTATTTGACACCA
CTGATTACCCCATTGATAGTCACACTTTGGGTTGTAAGTGACTTTTTATTTATTTG
TATTTTTGACTGCATTAAGAGGTCTCTAGTTTTTTATCTCTTGTTTCCCAAAACCT
AATAAGTAACTAATGCACAGAGCACATTGATTTGTATTTATTCTATTTTTAGACAT
AATTTATTAGCATGCATGAGCAAATTAAGAAAAACAACAACAAATGAATGCATA
TATATGTATATGTATGTGTGTATATATACACACATATATATATATATTTTTTCTTTT
CTTACCAGAAGGTTTTAATCCAAATAAGGAGAAGATATGCTTAGAACCGAGGTA
GAGTTTTCATCCATTCTGTCCTGTAAGTATTTTGCATATTCTGGAGACGCAGGAA
GAGATCCATCTACATATCCCAAAGCTGAATTATGGTAGACAAAACTCTTCCACTT
TTAGTGCATCAACTTCTTATTTGTGTAATAAGAAAATTGGGAAAACGATCTTCAA
TATGCTTACCAAGCTGTGATTCCAAATATTACGTAAATACACTTGCAAAGGAGGA
TGTTTTTAGTAGCAATTTGTACTGATGGTATGGGGCCAAGAGATATATCTTAGAG
GGAGGGCTGAGGGTTTGAAGTCCAACTCCTAAGCCAGTGCCAGAAGAGCCAAGG
ACAGGTACGGCTGTCATCACTTAGACCTCACCCtgtggagccacaccctagggttggccaatctactcc
caggagcagggagggcaggagccagggctgggcataaaagtcagggcagagccatctattgcttacatttgcttctgacacaactgt
gttcactagcaacctcaaacagacacc
SEQ ID NO: 20
HBB right homology arm
ggtctattttcccacccttaggctgctggtggtctacccttggacccagaggttctttgagtcctttggggatctgtccactcctgatgctgtt
atgggcaaccctaaggtgaaggctcatggcaagaaagtgctcggtgcctttagtgatggcctggctcacctggacaacctcaagggc
acctttgccacactgagtgagctgcactgtgacaagctgcacgtggatcctgagaacttcagggtgagtctatgggacgcttgatgtttt
ctttccccttcttttctatggttaagttcatgtcataggaaggggataagtaacagggtacagtttagaatgggaaacagacgaatgattgc
atcagtgtggaagtctcaggatcgttttagtttcttttatttgctgttcataacaattgttttcttttgtttaattcttgctttctttttttttcttctccgc
aatttttactattatacttaatgccttaacattgtgtataacaaaaggaaatatctctgagatacattaagtaacttaaaaaaaaactttacaca
gtctgcctagtacattactatttggaatatatgtgtgcttatttgcatattcataatctccctactttattttttttatttttaattgatacataatcatt
atacatatttatgggttaaagtgtaatgttttaatatgtgtacacatattgaccaaatcagggtaattttgcatttgtaattttaaaaaatgctttct
tcttttaatatacttttttgtttatcttatttctaatactttccctaatctctttctttcagggcaataatgatacaatgtatcatgcctctttgcaccatt
ctaaagaataacagtgataatttctgggttaaggcaa
SEQ ID NO: 21
HBB sg7 target sequence
5′-GGGTGGGAAAATAGACCAAT-3′
SEQ ID NO: 22
HBB sg11 target sequence
5′-TATGGTTAAGTTCATGTCAT-3′
SEQ ID NO: 23
HBB sg13 target sequence
5′-TAGGAAGGGGATAAGTAACA-3′
SEQ ID NO: 24
IRES sequence
cccctctccctcccccccccctaacgttactggccgaagccgcttggaataaggccggtgtgcgtttgtctatatgttattttccaccatatt
gccgtcttttggcaatgtgagggcccggaaacctggccctgtcttcttgacgagcattcctaggggtctttcccctctcgccaaaggaat
gcaaggtctgttgaatgtcgtgaaggaagcagttcctctggaagcttcttgaagacaaacaacgtctgtagcgaccctttgcaggcagc
ggaaccccccacctggcgacaggtgcctctgcggccaaaagccacgtgtataagatacacctgcaaaggcggcacaaccccagtg
ccacgttgtgagttggatagttgtggaaagagtcaaatggctctcctcaagcgtattcaacaaggggctgaaggatgcccagaaggta
ccccattgtatgggatctgatctggggcctcggtacacatgctttacatgtgtttagtcgaggttaaaaaaacgtctaggccccccgaac
cacggggacgtggttttcctttgaaaaacacgatgataatatggccacaacc
SEQ ID NO: 25
T2A cleavage peptide
CGCAAGCGCCGCAGCGGCAGCGGCGAGGGCCGCGGCAGCCTGCTGACCTGCGGC
GACGTGGAGGAGAACCCCGGCCCC
SEQ ID NO: 26
P2A cleavage peptide
CGCAAAAGACGCTCCGGTTCTGGAGAGGGCAGGGGGAGTCTTCTTACATGCGGG
GATGTTGAGGAGAATCCCGGACCC

What is claimed is:

1. A method of genetically modifying a hematopoietic stem and progenitor cell (HSPC), the method comprising:
introducing into the HSPC an RNA-guided nuclease and a guide RNA that specifically targets a sequence within the cytoplasmic domain-encoding region of the erythropoietin receptor (EPOR) locus in the genome of the cell; wherein
the RNA-guided nuclease cleaves the EPOR locus in the genome of the cell, resulting in the expression of a truncated erythropoietin receptor (tEPOR) in the cell and thereby generating a genetically modified HSPC; and wherein
the expression of the tEPOR increases the sensitivity of the cell to erythropoietin (EPO) and/or increases the proliferation of the cell in the presence of EPO as compared to the sensitivity and/or proliferation of a non-genetically modified HSPC.

2. The method of claim 1, wherein the cleavage of the EPOR locus by the RNA-guided nuclease creates an insertion or deletion (indel) that introduces a nonsense mutation into the EPOR locus.

3. The method of claim 1, further comprising introducing a donor template into the cell,
wherein the donor template comprises a first homologous region comprising complementarity to the EPOR locus upstream of the guide RNA target site, a second homologous region comprising complementarity to the EPOR locus downstream of the guide RNA target site, and a coding sequence located between the first and second homology regions that encodes a tEPOR, and wherein the coding sequence is integrated into the cleaved EPOR locus, leading to the expression of the tEPOR in the cell.

4. The method of claim 1, wherein the method further comprises isolating the HSPC from a subject prior to introducing the RNA-guided nuclease and the guide RNA into the cell.

5. The method of claim 1, wherein expression of the tEPOR is driven by the endogenous EPOR promoter.

6. The method of claim 1, wherein the tEPOR lacks a C-terminal portion of the EPOR cytoplasmic domain.

7. The method of claim 1, wherein the first homology arm comprises the nucleotide sequence of SEQ ID NO:1 or a subsequence thereof, or a sequence comprising at least about 70%, 75%, 80%, 85%, 90%, 95%, 96%, 97%, 98%, 99%, or more identity to SEQ ID NO:1 or a subsequence thereof.

8. The method of claim 1, wherein the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof.

9. The method of claim 1, wherein the coding sequence encoding the truncated EPOR protein comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10.

10. The method of claim 3, wherein the donor template comprises SEQ ID NO:3 or a subsequence thereof, or a sequence comprising at least about 70%, 75%, 80%, 85%, 90%, 95%, 96%, 97%, 98%, 99% or more identity to SEQ ID NO:3 or a subsequence thereof.

11. The method of claim 1, wherein the target sequence of the guide RNA comprises the nucleotide sequence of SEQ ID NO: 11 or 12, or a nucleotide sequence comprising 1, 2, or 3 mismatches relative to SEQ ID NO: 11 or 12.

12. The method of claim 1, wherein the guide RNA comprises one or more 2′-O-methyl-3′-phosphorothioate (MS) modifications.

13. The method of claim 12, wherein the one or more 2′-O-methyl-3′-phosphorothioate (MS) modifications are present at the three terminal nucleotides of the 5′ and 3′ ends of the guide RNA.

14. The method of claim 1, wherein the RNA-guided nuclease is Cas9.

15. The method of claim 14, wherein the Cas9 is a High Fidelity Cas9.

16. The method of claim 1, wherein the guide RNA and the RNA-guided nuclease are introduced into the HSPC as a ribonucleoprotein (RNP) complex by electroporation.

17. The method of claim 3, wherein the donor template is introduced into the HSPC using a recombinant adeno-associated virus (rAAV) vector.

18. The method of claim 17, wherein the rAAV vector is a AAV6 vector.

19. The method of claim 1, wherein the HSPC is genetically modified at a second locus other than EPOR, using an sgRNA targeting the second locus and a second homologous donor template comprising homology to the second locus.

20. The method of claim 19, wherein the second homologous donor template further comprises a therapeutic transgene.

21. The method of claim 20, wherein the therapeutic transgene is selected from the group consisting of HBA1, HBA2, HBB, PDGFB, IDUA, FIX, LDLR, and PAH.

22. The method of claim 19, wherein the HSPC is isolated from a subject having a condition for which the genetic modification made at the second locus is beneficial.

23. The method of claim 22, wherein the condition is α-thalassemia, β-thalassemia, sickle cell disease, hemophilia B, phenylketonuria, Gaucher disease, or Krabbe disease.

24. The method of claim 22, wherein the genetically modified HSPC is reintroduced into the subject.

25. The method of claim 24, wherein the reintroduction of the genetically modified HSPC ameliorates one or more symptoms of the condition.

26. The method of claim 24, wherein the proportion of genetically modified HSPCs among red blood cells (RBCs) and/or one or more myeloid or lymphoid lineages in the subject increases overtime.

27. The method of claim 4, wherein the subject is a human.

28. A method of genetically modifying a hematopoietic stem and progenitor cell (HSPC), the method comprising:
introducing into the HSPC an RNA-guided nuclease, a donor template comprising a transgene encoding a truncated erythropoictin receptor (tEPOR), and a guide RNA that specifically targets a sequence within a safe harbor locus in the genome of the cell; wherein
the donor template comprises a first homologous region comprising complementarity to the safe harbor locus upstream of the guide RNA target site, a second homologous region comprising complementarity to the safe harbor locus downstream of the guide RNA target site, wherein the first and second homology regions flank the tEPOR transgene on the template, wherein
the RNA-guided nuclease cleaves the safe harbor locus in the genome of the cell and the transgene is integrated into the genome at the cleaved safe harbor locus, thereby generating a genetically modified HSPC; and wherein
the integrated transgene results in expression of the tEPOR in the genetically modified HSPC.

29. The method of claim 28, wherein expression of the tEPOR increases the sensitivity of the cell to erythropoietin (EPO) and/or increases the proliferation of the cell in the presence of EPO relative to the sensitivity and/or proliferation of a non-genetically modified HSPC.

30. The method of claim 28, wherein the method further comprises isolating the HSPC from a subject prior to introducing the RNA-guided nuclease, the donor template, and the guide RNA into the cell.

31. The method of claim 28, wherein the transgene encoding the truncated EPOR comprises the nucleotide sequence of SEQ ID NO:10, or a sequence comprising at least about 70%, 75%, 80%, 85%, 90%, 95%, 96%, 97%, 98%, 99%, or more identity to SEQ ID NO:10.

32. The method of claim 28, wherein the guide RNA comprises one or more 2′-O-methyl-3′-phosphorothioate (MS) modifications.

33. The method of claim 32, wherein the one or more 2′-O-methyl-3′-phosphorothioate (MS) modifications are present at the three terminal nucleotides of the 5′ and 3′ ends of the guide RNA.

34. The method of claim 28, wherein the RNA-guided nuclease is Cas9.

35. The method of claim 34, wherein the Cas9 is a High Fidelity Cas9.

36. The method of claim 28, wherein the guide RNA and the RNA-guided nuclease are introduced into the HSPC as a ribonucleoprotein (RNP) complex by electroporation.

37. The method of claim 28, wherein the donor template is introduced into the HSPC using a recombinant adeno-associated virus (rAAV) vector.

38. The method of claim 37, wherein the rAAV vector is a AAV6 vector.

39. The method of claim 28, wherein the safe harbor locus is CCR5.

40. The method of claim 39, wherein the first homology arm comprises the nucleotide sequence of SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof.

41. The method of claim 39, wherein the second homology arm comprises the nucleotide sequence of SEQ ID NO:8 or a subsequence thereof, or a sequence comprising at least about 70%, 75%, 80%, 85%, 90%, 95%, 96%, 97%, 98%, 99%, or more identity to SEQ ID NO:8 or a subsequence thereof.

42. The method of claim 39, wherein the target sequence of the guide RNA comprises the nucleotide sequence of SEQ ID NO: 14, or a nucleotide sequence comprising 1, 2, or 3 mismatches relative to SEQ ID NO: 14.

43. The method of claim 39, wherein the donor template comprises SEQ ID NO:9 or a subsequence thereof, or a sequence comprising at least about 70%, 75%, 80%, 85%, 90%, 95%, 96%, 97%, 98%, 99% or more identity to SEQ ID NO:9 or a subsequence thereof.

44. The method of claim 28, wherein the safe harbor locus is HBA1.

45. The method of claim 44, wherein the first homology arm comprises the nucleotide sequence of SEQ ID NO:4 or a subsequence thereof, or a sequence comprising at least about 70%, 75%, 80%, 85%, 90%, 95%, 96%, 97%, 98%, 99%, or more identity to SEQ ID NO:4 or a subsequence thereof.

46. The method of claim 44, wherein the second homology arm comprises the nucleotide sequence of SEQ ID NO:5 or a subsequence thereof, or a sequence comprising at least about 70%, 75%, 80%, 85%, 90%, 95%, 96%, 97%, 98%, 99%, or more identity to SEQ ID NO:5 or a subsequence thereof.

47. The method of claim 44, wherein the target sequence of the guide RNA comprises the nucleotide sequence of SEQ ID NO: 13, or a nucleotide sequence comprising 1, 2, or 3 mismatches relative to SEQ ID NO: 13.

48. The method of claim 44, wherein the donor template comprises SEQ ID NO:6 or a subsequence thereof, or a sequence comprising at least about 70%, 75%, 80%, 85%, 90%, 95%, 96%, 97%, 98%, 99% or more identity to SEQ ID NO:6 or a subsequence thereof.

49. The method of claim 44, wherein the donor template further comprises a therapeutic transgene encoding a protein, and wherein the first and second homology regions flank the therapeutic transgene and the tEPOR transgene on the template.

50. The method of claim 49, wherein the donor template further comprises an internal ribosome entry site (IRES) or a sequence encoding a 2A cleavage peptide between the therapeutic transgene and the tEPOR transgene on the template.

51. The method of claim 49, wherein the HSPC comprises a mutation in an endogenous gene causative of a condition in a subject and the therapeutic transgene comprises a corrective sequence.

52. The method of claim 49, wherein the therapeutic transgene is selected from the group consisting of HBB, PDGFB, IDUA, FIX, LDLR, and PAH.

53. The method of claim 51, wherein the therapeutic transgene is HBB and the condition is β-thalassemia or sickle cell disease.

54. The method of claim 51, wherein the therapeutic transgene is FIX and the condition is hemophilia B.

55. The method of claim 51, wherein the therapeutic transgene is PAH and the condition is phenylketonuria.

56. The method claim 49, wherein the HSPC comprises a population of HSPCs.

57. The method of claim 56, wherein expression of the therapeutic transgene and the tEPOR transgene causes an enrichment of genetically modified HSPCs in the population of HSPCs over the course of red blood cell differentiation as compared to expression of the therapeutic transgene in the absence of expression of the tEPOR transgene.

58. The method of claim 49, wherein expression of the therapeutic transgene and the tEPOR transgene increases a level of adult hemoglobin tetramers in the genetically modified HSPC as compared to expression of the therapeutic transgene in the absence of expression of the tEPOR transgene.

59. The method of claim 28, wherein the safe harbor locus is HBB.

60. The method of claim 59, wherein the first homology arm comprises the nucleotide sequence of SEQ ID NO:19 or a subsequence thereof, or a sequence comprising at least about 70%, 75%, 80%, 85%, 90%, 95%, 96%, 97%, 98%, 99%, or more identity to SEQ ID NO:19 or a subsequence thereof.

61. The method of claim 59, wherein the second homology arm comprises the nucleotide sequence of SEQ ID NO:20 or a subsequence thereof, or a sequence comprising at least about 70%, 75%, 80%, 85%, 90%, 95%, 96%, 97%, 98%, 99%, or more identity to SEQ ID NO:20 or a subsequence thereof.

62. The method of claim 59, wherein the target sequence of the guide RNA comprises the nucleotide sequence of SEQ ID NO: 21, or a nucleotide sequence comprising 1, 2, or 3 mismatches relative to SEQ ID NO: 21.

63. The method of claim 59, wherein the target sequence of the guide RNA comprises the nucleotide sequence of SEQ ID NO: 22 or 23, or a nucleotide sequence comprising 1, 2, or 3 mismatches relative to SEQ ID NO: 22 or 23.

64. The method of claim 59, wherein the donor template further comprises a therapeutic transgene encoding a protein, and wherein the first and second homology regions flank the therapeutic transgene and the tEPOR transgene on the template.

65. The method of claim 64, wherein the donor template further comprises an internal ribosome entry site (IRES) or a sequence encoding a 2A cleavage peptide between the therapeutic transgene and the tEPOR transgene on the template.

66. The method of claim 64, wherein the HSPC comprises a mutation in an endogenous gene causative of a condition in a subject and the therapeutic transgene comprises a corrective sequence.

67. The method of claim 64, wherein the therapeutic transgene is HBA1 or HBA2.

68. The method of claim 66, wherein the condition is α-thalassemia.

69. The method of claim 64, wherein the HSPC comprises a population of HSPCs.

70. The method of claim 69, wherein expression of the therapeutic transgene and the tEPOR transgene causes an enrichment of genetically modified HSPCs in the population of HSPCs over the course of red blood cell differentiation as compared to expression of the therapeutic transgene in the absence of expression of the tEPOR transgene.

71. The method of claim 64, wherein expression of the therapeutic transgene and the tEPOR transgene increases a level of adult hemoglobin tetramers in the genetically modified HSPC as compared to expression of the therapeutic transgene in the absence of expression of the tEPOR transgene.

72. The method of claim 28, wherein the HSPC is genetically modified at a second locus other than the safe harbor locus using an sgRNA targeting the second locus and a second homologous donor template comprising homology to the second locus.

73. The method of claim 72, wherein the second homologous donor template further comprises a therapeutic transgene.

74. The method of claim 73, wherein the therapeutic transgene is selected from the group consisting of HBA1, HBA2, HBB, PDGFB, IDUA, FIX, LDLR, and PA H.

75. The method of claim 72, wherein the HSPC is isolated from a subject having a condition for which the genetic modification introduced at the second locus is beneficial.

76. The method of claim 75, wherein the condition is α-thalassemia, β-thalassemia, sickle cell disease, hemophilia B, phenylketonuria, Gaucher disease, or Krabbe disease.

77. The method of claim 51, wherein the genetically modified HSPC is reintroduced into the subject.

78. The method of claim 77, wherein the reintroduction of the genetically modified HSPC ameliorates one or more symptoms of the condition.

79. The method of claim 77, wherein the proportion of genetically modified HSPCs among red blood cells (RBCs) and/or one or more myeloid or lymphoid lineages in the subject increases over time following the reintroduction of the HSPC into the subject.

80. The method of claim 28, wherein the transgene comprises a heterologous promoter.

81. The method of claim 80, wherein the heterologous promoter is selected from the group consisting of EPOR, HBA1, PGK1, and UBC.

82. The method of claim 30, wherein the subject is a human.

83. A genetically modified HSPC comprising a coding sequence encoding a tEPOR, wherein the genetically modified HSPC is generated using the method of any one of claims 1 to 82.

84. A donor template comprising a homology region comprising SEQ ID NO: 1 or SEQ ID NO: 2 or a subsequence thereof, or a nucleotide sequence comprising at least about 70%, 75%, 80%, 85%, 90%, 95%, 96%, 97%, 98%, 99% or more identity to SEQ ID NO: 1 or SEQ ID NO: 2 or a subsequence thereof.

85. A donor template comprising a nucleotide sequence selected from the group consisting of SEQ ID NOS: 3, 6, and 9 and subsequences thereof, or a nucleotide sequence comprising at least about 70%, 75%, 80%, 85%, 90%, 95%, 96%, 97%, 98%, 99% or more identity to any one of SEQ ID NOS: 3, 6, or 9, or a subsequence thereof.

86. A transgene comprising a nucleotide sequence encoding a tEPOR, wherein the nucleotide sequence comprises the sequence of SEQ ID NO:10, or a nucleotide sequence comprising at least about 70%, 75%, 80%, 85%, 90%, 95%, 96%, 97%, 98%, 99% or more identity to SEQ ID NO:10.

87. A guide RNA comprising a target sequence comprising SEQ ID NO: 11 or SEQ ID NO:12, or a sequence comprising 1, 2, or 3 mismatches with SEQ ID NO: 11 or SEQ ID NO:12.

88. An HSPC comprising the donor template of claim 84 or 85, the transgene of claim 86, and/or the guide RNA of claim 87.
